(19) United States
(12) Patent Application Publication
(10) Pub. No.: US 2026/0207640 A1
(43) Pub. Date: Jul. 23, 2026

(54) Polyphenolic Boronates and Use Thereof

(71) Applicants: The Medical College of Wisconsin, Inc., Milwaukee, WI (US); Aix-Marseille Université, Marseille (FR)

(72) Inventors: Balaraman Kalyanaraman, Milwaukee, WI (US); Micael Joël Hardy, Nimes (FR); Gang Cheng, Libertyville, IL (US)

(21) Appl. No.: 19/140,159

(22) PCT Filed: Dec. 11, 2023
(86) PCT No.: PCT/US2023/083436

§ 371 Date: Jun. 17, 2025

Related U.S. Application Data
(60) Provisional application No. 63/476,341, filed on Dec. 20, 2022.

(51) Int. Cl. A61K 31/69; A61P 35/00; C07F 5/02

(52) U.S. Cl. CPC A61K 31/69; A61P 35/00; C07F 5/025

(57) ABSTRACT

The present disclosure provides polyphenolic boronates and mitochondria-targeted polyphenolic boronates and pharmaceutical compositions thereof. The present compounds may be useful for treating cancer and reducing or inhibiting cancer cell growth. The present compounds may be useful for reducing or analyzing reactive oxygen and nitrogen species in tumor microenvironment of a cancer. The present compounds may target mitochondria in carcer cells and have improved anti-tumor effect and reduced toxicity.

### CROSS-REFERENCE TO RELATED APPLICATIONS

The present application claims priority to U.S. Provisional Patent Application No. 63/476,341, filed Dec. 20, 2022, the content of which is hereby incorporated by reference in its entirety.

### STATEMENT REGARDING FEDERALLY SPONSORED RESEARCH OR DEVELOPMENT

Not Applicable.

### BACKGROUND

Boronate-based compounds have been used in brain cancer therapy, either as prodrugs or in combination with other modalities. Boronates containing pro-luminescent and fluorescent probes have been used in mouse models of cancer. Borax and boronophenylalanine have been used in boron neutron capture therapy (BNCT) to treat glioma. Literature has indicated the antiproliferative and antitumor effects of several boron-containing compounds in various cancers. In some studies, boronates are used as prodrugs, releasing the active antitumor drug in situ, which occurs following its reaction with hydrogen peroxide (H₂O₂). Boronate-based fluorophores and positron-emission tomography (PET) active compounds have been in cancer cells and cancer xenografts in the detection of oxidants.

There remains a need for boronate-based compounds that have improved properties, including increased potency and reduced cell toxicity, that are effective in inhibiting cancer cell proliferation and can be used for immune therapies.

### SUMMARY OF THE INVENTION

Disclosed herein are polyphenolic boronate and mitochondria-targeted polyphenolic boronate compounds, pharmaceutical compositions comprising the compounds, kits, and methods of use thereof.

In one aspect, the present disclosure provides a compound of formula (I) or (II),

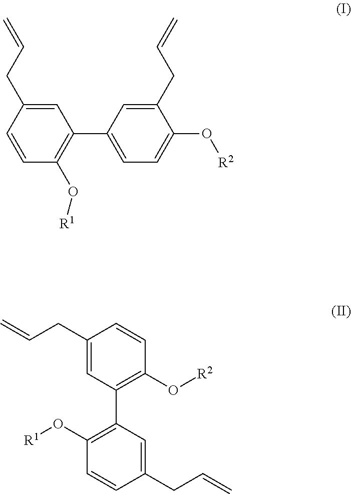

wherein
one of R¹ and R² is H,

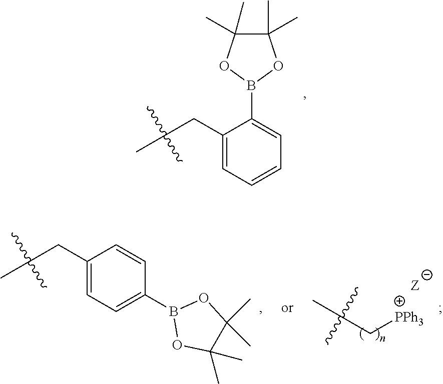

the other of R¹ and R² is

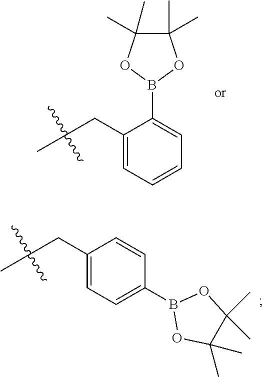 [SMILES: *Cc1ccccc1B1OC(C)(C)C(C)(C)O1 ; *Cc1ccc(B2OC(C)(C)C(C)(C)O2)cc1]

n is an integer from 1-20; and
Z^{⊖} is a counterion.

In another aspect, the present disclosure provides a pharmaceutical composition. The pharmaceutical composition comprises the compound as disclosed herein, and a pharmaceutically acceptable carrier, diluent, or excipient.

In a further aspect, the present disclosure provides a method of treating cancer in a subject in need thereof. The method comprises administering to the subject a therapeutically effective amount of the compound as described herein, or the pharmaceutical composition as described herein.

In another aspect, the present disclosure provides a method of reducing reactive oxygen and nitrogen species in tumor microenvironment of a cancer in a subject in need thereof. The method comprises administering to the subject an effective amount of the compound as described herein, or the pharmaceutical composition as described herein.

In another aspect, the present disclosure provides a method of analyzing reactive oxygen or nitrogen species in tumor microenvironment of a cancer. The method comprises: introducing the compound as described herein or the pharmaceutical composition as described herein to the tumor microenvironment, whereby the reactive oxygen or nitrogen species reacts with the compound or composition to produce a product, and detecting the product thereby analyzing the reactive oxygen or nitrogen species in the tumor microenvironment.

In another aspect, the present disclosure provides a method of reducing or inhibiting cancer cell growth in a subject in need thereof. The method comprises administering a therapeutically effective amount of the compound as described herein or the pharmaceutical composition as described herein to the subject.

In a further aspect, the present disclosure provides a compound or a pharmaceutical composition for use in therapy.

In another aspect, the present disclosure provides a compound or a pharmaceutical composition for use in treatment of cancer.

In another aspect, the present disclosure provides a compound or a pharmaceutical composition for use in boron neutron capture therapy (BNCT) for cancer.

In some embodiments, the present compound or pharmaceutical composition is targeted to cancer cell mitochondria.

In yet another aspect, the present disclosure provides use of the compound as described herein for the manufacture of a medicament for the treatment of cancer.

In yet another aspect, the present disclosure provides a kit. The kit comprises the pharmaceutical composition as described herein and an instructional material.

### BRIEF DESCRIPTION OF THE DRAWINGS

FIG. 1 shows structures of selected polyphenolic boronates and polyphenols, and the calculated values of the octanol/water partition coefficients.

FIGS. 2A-2C show effects of HNK and HNK-B on the proliferation of glioblastoma (U87MG), pancreatic cancer (MiaPaCa-2) cells, and lung cancer (A549) cells. The effects of HNK and HNK-B on the proliferation of U87MG (FIG. 2A), MiaPaCa-2 (FIG. 2B), or A549 (FIG. 2C) cells were monitored in the IncuCyte Live-Cell Analysis system. The IncuCyte analyzer provides real-time updates on cell confluence based on segmentation of high definition-phase contrast images. Representative cell images are shown as segmentation masks, illustrated when control cells reached 90% confluence (vertical solid line). The half maximal inhibitory concentration (IC50) values for U87MG (FIG. 2A), MiaPaCa-2 (FIG. 2B), or A549 (FIG. 2C) cells were determined at the point at which control cells reached ~90% confluence (vertical solid line). Relative cell confluence (control is taken as 100%) is plotted against concentration. Dashed lines represent the fitting curves used to determine the IC₅₀ values as indicated. Data shown are the mean±standard deviation (SD).

FIGS. 3A-3C demonstrate effects of Mito-HNK and Mito-HNK-B on the proliferation of glioblastoma (U87MG), pancreatic cancer (MiaPaCa-2), and lung cancer (A549) cells. The effects of Mito-HNK and Mito-HNK-B on the proliferation of U87MG (FIG. 3A), MiaPaCa-2 (FIG. 3B), or A549 (FIG. 3C) cells were monitored in the IncuCyte Live-Cell Analysis system. The IncuCyte analyzer provides real-time updates on cell confluence based on segmentation of high definition-phase contrast images. Representative cell images are shown as segmentation masks, illustrated when control cells reached 90% confluence (vertical solid line). The IC50 values for U87MG (FIG. 3A), MiaPaCa-2 (FIG. 3B), or A549 (FIG. 3C) cells were determined at the point at which control cells reached ~90% confluence (vertical solid line). Relative cell confluence (control is taken as 100%) is plotted against concentration. Dashed lines represent the fitting curves used to determine the IC50 values as indicated. Data shown are the mean±SD.

FIGS. 4A-4B show effects of Mito-MGN, Mito-MGN-B, MGN, and MGN-B on the proliferation of glioblastoma cancer (U87MG) cells. The effects of Mito-MGN, Mito-MGN-B, MGN, and MGN-B on the proliferation of U87MG cells were monitored in the IncuCyte Live-Cell Analysis system. The IncuCyte analyzer provides real-time updates on cell confluence based on segmentation of high definition-phase contrast images. Representative cell images are shown as segmentation masks, illustrated in black when control cells reached 90% confluence. Full growth curves of Mito-MGN and Mito-MGN-B are shown in panel (FIG. 4A). The IC50 values were determined at the point at which control cells reached ~90% confluence. Relative cell confluence (control is taken as 100%) is plotted against concentration. Dashed lines in FIG. 4B represent the fitting curves used to determine the IC₅₀ values as indicated for Mito-MGN and Mito-MGN-B (left panel) and for MGN and MGN-B (right panel). Data shown are the mean±SD.

FIGS. 5A-5B show effects of MGN and MGN-B on the proliferation of melanoma cancer (UACC-62) cells. The effects of MGN and MGN-B on the proliferation of UACC-62 cells were monitored in the IncuCyte Live-Cell Analysis system. The IncuCyte analyzer provides real-time updates on cell confluence based on segmentation of high definition-phase contrast images. Full growth curves of MGN and MGN-B are shown in FIG. 5A. The IC₅₀ values were determined at the point at which control cells reached ~90% confluence. Relative cell confluence (control is taken as 100%) is plotted against concentration. Dashed lines in FIG. 5B represent the fitting curves used to determine the IC₅₀ values as indicated for MGN and MGN-B. Data shown are the mean±SD.

FIGS. 6A-6C demonstrate HPLC traces of the reaction between oxidants and HNK derivatives. FIG. 6A shows reaction of HNK-B (500 M) in the presence of H₂O₂, NaOCl, and ONOO− (500 μM). FIG. 6B shows reaction of HNK-B² (500 μM) in the presence of H₂O₂ (500 μM and 2 mM) and ONOO− (500 μM). FIG. 6C shows reaction of Mito-HNK-B (500 μM) in the presence of H₂O₂, NaOCl, and ONOO− (500 μM). All samples were incubated at room temperature in aqueous solution containing phosphate buffer (0.1 M, pH 7.4) and DTPA (10 μM). Absorption traces recorded using UV absorption detection at 260 nm.

FIGS. 7A-7C demonstrate structures, mass spectral parameters, and retention times of reactants, intermediates, and products formed from the reaction between oxidants and HNK derivatives. FIG. 7A shows products formed from the reaction of HNK-B with oxidants. FIG. 7B shows products in the presence of HNK-B². FIG. 7C shows products in the presence of Mito-HNK-B.

FIG. 8 shows oxidant-induced transformation of polyphenolic boronates to corresponding phenols.

FIG. 9 illustrates a proposed antiproliferative and antioxidant potential of polyphenolic boronates in tumor mitochondria and the TME. One of the mechanisms of immunosuppression in the TME is myeloid cell-induced generation of reactive oxygen species and reactive nitrogen species (H₂O₂ and ONOO−). Boronates can remove reactive oxygen species and reactive nitrogen species and activate T cells in addition to generating polyphenols and mitochondria-targeted polyphenols in situ in the tumor microenvironment.

### DETAILED DESCRIPTION OF THE INVENTION

Before the present materials and methods are described, it is understood that this invention is not limited to the particular methodology, protocols, materials, and reagents described, as these may vary. It is also to be understood that the terminology used herein is for the purpose of describing particular embodiments only and is not intended to limit the scope of the present invention which will be limited only by the appended claims.

As used in this specification and the claims, the singular forms “a,” “an,” and “the” include plural forms unless the context clearly dictates otherwise. For example, the term “a compound” should be interpreted to mean “one or more compounds” unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkyl” as used herein, means a straight or branched chain saturated hydrocarbon. The alkyl can be a C₁₋₄alkyl. Representative examples of alkyl include, but are not limited to, methyl, ethyl, n-propyl, isopropyl, n-butyl, sec-butyl, isobutyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylene,” as used herein, means a divalent group derived from a straight or branched chain saturated hydrocarbon. Representative examples of alkylene include, but are not limited to, —CH₂—, —CH₂CH₂—, —CH₂CH₂CH₂—, —CH₂CH(CH₃)CH₂—, and CH₂CH(CH₃)CH(CH₃)CH₂—.

The term “alkene” as used herein, means an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively.

The term “alkenylene” as used herein, means a divalent group derived from a straight or branched alkene, which attaches to the parent molecule at two different carbon atoms.

The term “aryl,” as used herein, means a carbocyclic aromatic group (e.g., phenyl or a bicyclic aryl). The term “aryl” includes polycyclic ring systems having one or more carbocyclic rings in which two or more carbons are common to two adjoining rings (the rings are “fused rings”) wherein at least one of the rings is aromatic and, e.g., the other ring(s) may be cycloalkyls or cycloalkenyls. For example, a bicyclic aryl can be a phenyl fused to a cycloalkyl moiety. Examples of aryl include naphthyl, dihydronaphthalenyl, tetrahydronaphthalenes, indanyl, or indenyl. The aryl (e.g., phenyl and bicyclic aryls) is attached to the parent molecular moiety through any carbon atom contained within the aryl.

The term “arylene” as used herein, means a divalent group derived from an aryl as described herein, which attaches to the parent molecule at two different ring carbon atoms. Examples of arylene includes, but are not limited to, phenylene, which is a divalent group derived from benzene and attaches to the parent molecule at two different ring carbon atoms (e.g., at 1,2-, 1,3-, or 1,4-positions).

The term “cycloalkyl” as used herein, means a monovalent group derived from an all-carbon ring system containing zero heteroatoms as ring atoms, and zero double bonds. The all-carbon ring system can be a monocyclic, bicylic, or tricyclic ring system, and can be a fused ring system, a bridged ring system, or a spiro ring system, or combinations thereof. Examples of cycloalkyls include, but are not limited to, cyclopropyl, cyclobutyl, cyclopentyl, cyclohexyl, cycloheptyl, cyclooctyl, and

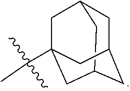 [SMILES: *C12CC3CC(CC(C3)C1)C2]

The cycloalkyl groups described herein can be appended to the parent molecular moiety through any substitutable carbon atom.

The term “cycloalkylene” as used herein, means a divalent group derived from an all-carbon ring system containing zero heteroatoms as ring atoms and zero double bonds, which attaches to the parent molecule at two different ring carbons atoms. The all-carbon ring system can be a monocyclic, bicylic, or tricyclic ring system, and can be a fused ring system, a bridged ring system, or a spiro ring system. Representative examples of cycloalkylene include, but are not limited to, those derived from C₃₋₁₀ rings, such as

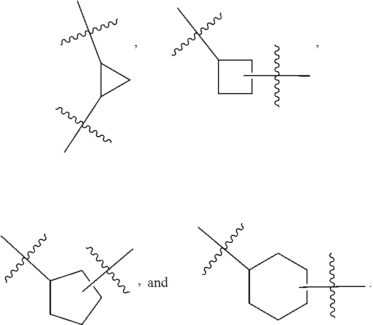

The term “halogen” or “halo” means a chlorine, bromine, iodine, or fluorine atom.

Terms such as “alkyl,” “cycloalkyl,” “alkylene,” “arylene,” or “cycloalkylene,” etc. may be preceded by a designation indicating the number of atoms present in the group in a particular instance (e.g., “C₁-C₄alkyl,” “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁-C₄” or “C₁₋₄,” the members of the group that follows may have any number of carbon atoms falling within the recited range. A “C₁-C₄alkyl” or “C₁₋₄alkyl,” for example, is an alkyl group having from 1 to 4 carbon atoms, however arranged (i.e., straight chain or branched).

If substituents are described as being independently selected from a group, each substituent is selected independent of the other. Each substituent, therefore, may be identical to or different from the other substituent(s).

The term “counterion” as used herein, means an anion that has a −1 charge. Common counterions include, but are not limited to, halide (e.g., F⁻, Cl⁻, Br⁻, I⁻), acetate (e.g., CH₃C(O)O⁻), monofluoroacetate, difluoroacetate, trifluoroacetate (e.g., CF₃C(O)O⁻), nitrate (e.g., NO₃⁻), nitrite (e.g., NO₂⁻), etc.

Unless otherwise stated, structures depicted herein are also meant to include all isomeric (e.g., enantiomeric, diastereomeric, regioisomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. The compound disclosed herein may exist as a regioisomer or a mixture of regioisomers. Unless otherwise stated, all tautomeric and regioisomeric forms of the compounds of the invention are within the scope of the invention.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

### Compounds

It has been known for decades that boronates react stoichiometrically yet slowly (k=1 M⁻¹s⁻¹) with H₂O₂, forming the corresponding hydroxyl product. Boron is electron-deficient and a weak Lewis acid. Biologically relevant proinflammatory oxidants such as peroxynitrite (ONOO⁻), hypochlorous acid (HOCl), and H₂O₂ have been shown to undergo a nucleophilic addition reaction with phenylboronates and stoichiometrically form the corresponding phenol as a major product. An interesting finding is that ONOO⁻ reacts nearly a million times faster with boronates than H₂O₂. Based on the rate constant determinations, boronates can compete very effectively with other biological reductants, such as glutathione, in their ability to scavenge ONOO⁻.

The structures of honokiol and magnolol, the two active components of magnolia extract, were modified. Boronate moieties were conjugated to naturally occurring polyphenolic compounds and their mitochondria-targeted analogs. These boronate-based polyphenolics can react with proinflammatory oxidants such as ONOO⁻, HOCl, and H₂O₂, and generate the original compounds in situ.

In one aspect, the present disclosure provides a compound of formula (I) or (II),

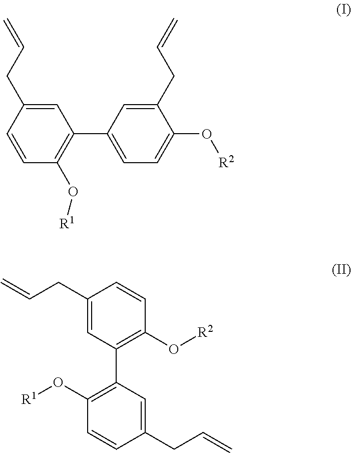

wherein
one of R¹ and R² is H,

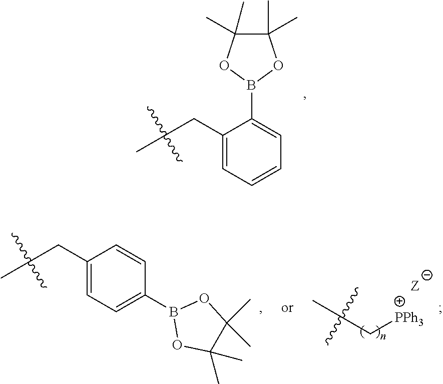

the other of R¹ and R² is

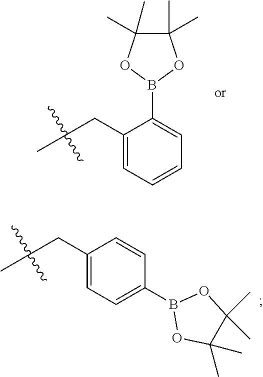 [SMILES: *Cc1ccccc1B1OC(C)(C)C(C)(C)O1 ; *Cc1ccc(B2OC(C)(C)C(C)(C)O2)cc1]

n is an integer from 1-20; and
Z is a counterion.

In some embodiments, the compound is a compound of formula (I). In some embodiments, the compound is a compound of formula (I) and R¹ is selected from the group consisting of H,

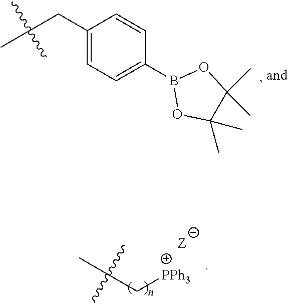

In some embodiments, the compound is compound of formula (I) and R¹ is selected from the group consisting of H,

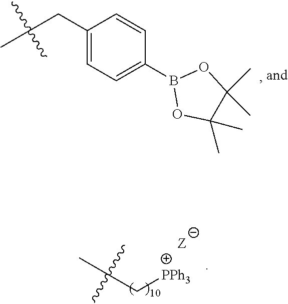

In some embodiments, the compound is a compound of formula (I) and R¹ is

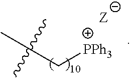

In some embodiments, the compound is a compound of formula (I) and R² is selected from the group consisting of H,

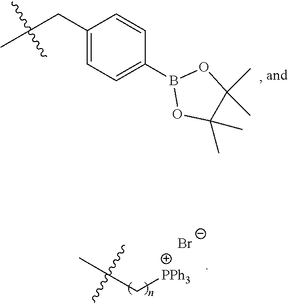

In some embodiments, the compound is a compound of formula (I) and R² is selected from the group consisting of H,

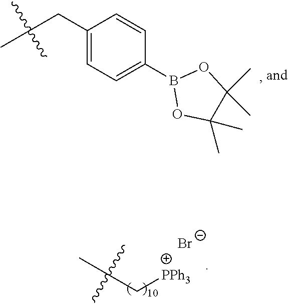

In some embodiments, the compound is a compound of formula (I) and R² is

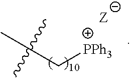

In some embodiments, the compound is a compound of formula (II). In some embodiments, the compound is a compound of formula (II) and R¹ selected from the group consisting of H,

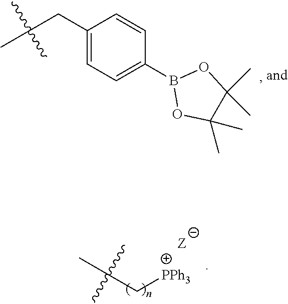

In some embodiments, the compound is a compound of formula (II) and R¹ is selected from the group consisting of H,

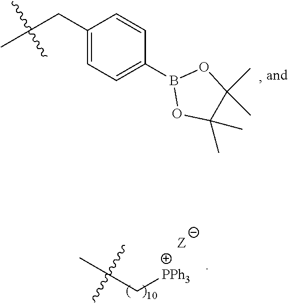

In some embodiments, the compound is a compound of formula (II) and R¹ is

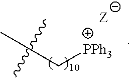

In some embodiments, one of R¹ and R² is

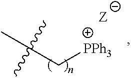

and the other of R¹ and R² is

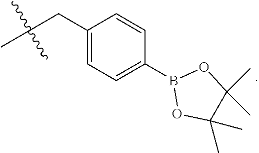 [SMILES: *Cc1ccc(B2OC(C)(C)C(C)(C)O2)cc1]

In some embodiments, one of R¹ and R² is

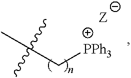

and the other of R¹ and R² is

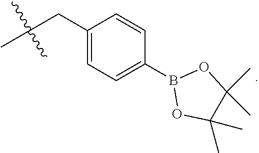 [SMILES: *Cc1ccc(B2OC(C)(C)C(C)(C)O2)cc1]

In some embodiments, n is an integer from 1-10. In some embodiments, n is an integer from 10-20. In some embodiments, n is an integer from 3 to 17, from 5 to 15, or from 7 to 12. In some embodiments, n is 6, 8, 10, 12, or 14. In some embodiments, n is 10.

Z can be a pharmaceutically acceptable counterion. In some embodiments, Z is Br, acetate, or trifluoroacetate. In some embodiments, Z is Br.

In some embodiments, the compound is

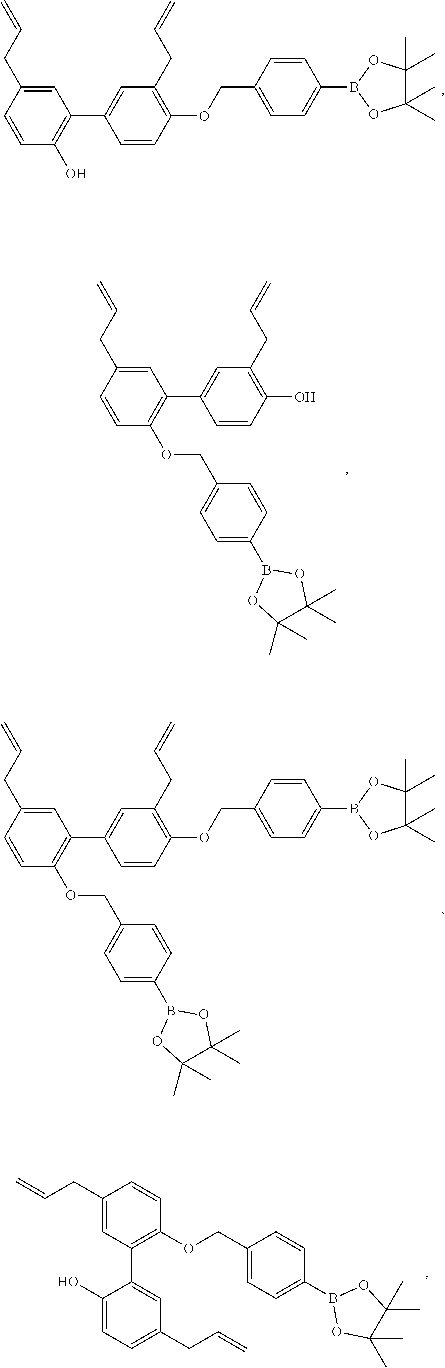

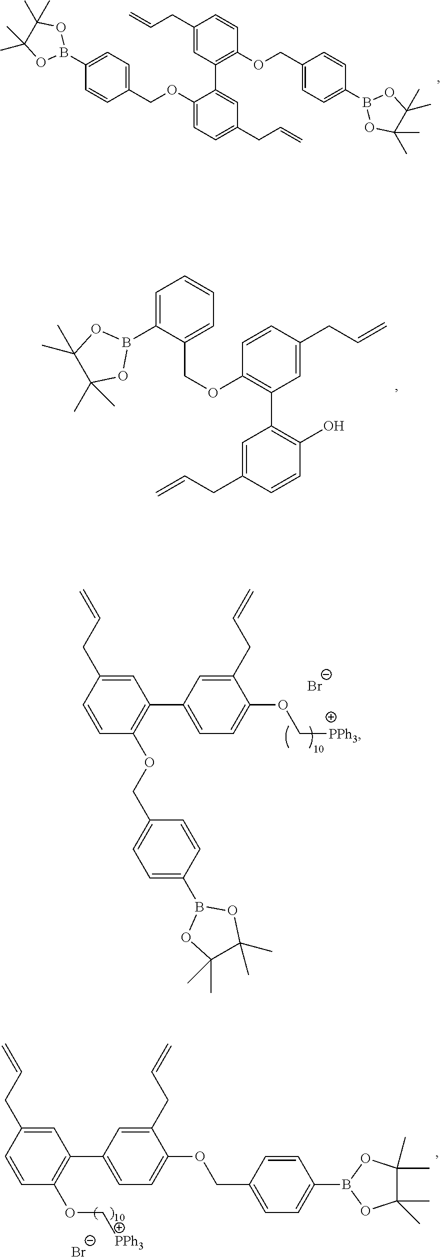

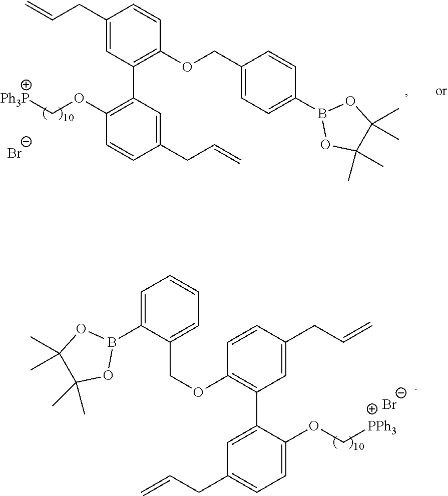

In some embodiments, the compound is

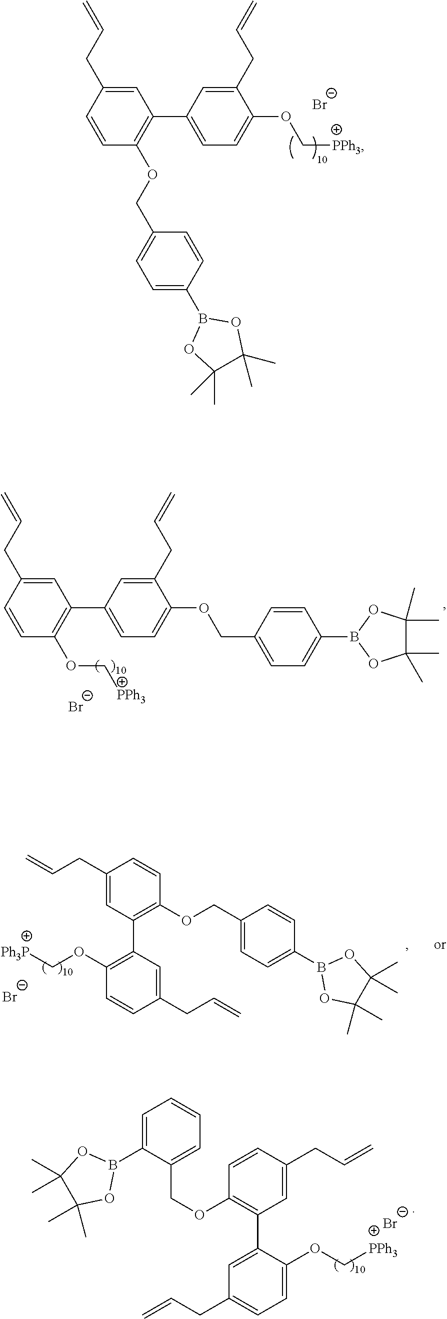

### Pharmaceutical Compositions

Another aspect of the disclosure provides a pharmaceutical composition. The pharmaceutical composition comprises the compound as described herein, and a pharmaceutically acceptable carrier, diluent, or excipient.

The pharmaceutical composition may include the compound in a range of about 0.1 to 2000 mg. In some embodiments, the pharmaceutical composition may include the compound in a range of from about 0.5 to 500 mg. In some embodiments, the pharmaceutical composition may include the compound in a range of from about 1 to 100 mg. The pharmaceutical composition may be administered to provide the compound at a daily dose of about 0.1 to about 1000 mg/kg body weight. In some embodiments, the pharmaceutical composition may be administered to provide the compound at a daily dose of about 0.5 to about 500 mg/kg body weight. In some embodiments, the pharmaceutical composition may be administered to provide the compound at a daily dose of about 50 to about 100 mg/kg body weight. In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 μM, 0.005 μM, 0.01 μM, 0.5 μM, 0.1 μM, 1.0 μM, 10 μM, and 100 μM (e.g., 0.1 μM-1.0 μM).

The compounds may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are in some embodiments applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

Optionally, the disclosed compounds or pharmaceutical compositions comprising the disclosed compounds may be administered with additional therapeutic agents, optionally in combination, in order to treat cancers. In some embodiments of the disclosed methods, one or more additional therapeutic agents are administered with the disclosed compounds or with pharmaceutical compositions comprising the disclosed compounds, where the additional therapeutic agent is administered prior to, concurrently with, or after administering the disclosed compounds or the pharmaceutical compositions comprising the disclosed compounds. In some embodiments, the disclosed pharmaceutical compositions are formulated to comprise the disclosed compounds and further to comprise one or more additional therapeutic agents, for example, one or more additional therapeutic agents for treating cancers.

### Methods of Use

There is increasing interest in understanding the role of oxidant formation and nitrosative stress in immunosuppressive tumor microenvironment (TME). Mitigating nitrosative stress has been reported to activate cytotoxic T cells involved in killing tumor cells. Inhibition of cytokine nitration induced by reactive nitrogen species eliminated T cell exhaustion. Reports also suggest that enhancing oxidant formation is critical for enhancing immunotherapy.

The compounds as described herein, which include honokiol boronate and mitochondria-targeted honokiol boronate, can target the mitochondria of tumor cells and inhibit cell proliferation. These boronate derivatives also can react with oxidants that are generated in tumor mitochondria and the tumor microenvironment. During this process, these boronate derivatives can be converted back to the original compounds with antitumor potencies. Thus, boronation of naturally occurring plant-derived compounds can make them more active in tumor cells as well as in the adjoining tumor microenvironment. These polyphenolic derivatives may enhance the scope of cancer immunotherapies. In particular, these boronate-based polyphenolics can react with proinflammatory oxidants such as ONOO⁻, HOCl, and H₂O₂, and generate the original compounds in situ. These polyphenolics exert an anti-immune and antitumor function. In some cases, polyphenolic boronates themselves exhibit more potency than the parent polyphenolics. In addition, these compounds can stoichiometrically scavenge reactive oxygen and nitrogen species generated in tumors and in the TMEs from immune cells (e.g., neutrophils, macrophages). Thus, the present compounds incorporating the oxidative phosphorylation (OXPHOS)-inhibiting drug group attached to a boronate may decrease T cell exhaustion through scavenging of nitric oxide-derived oxidants released in the TME, thereby generating a polyphenolic mitochondria-targeted drug (MTD) with antitumor function.

### Method of Treating Cancer

In another aspect, the present disclosure provides a method of treating cancer in a subject in need thereof. The method comprises administering to the subject a therapeutically effective amount of the compound as disclosed herein, or the pharmaceutical composition as disclosed herein. In one embodiment, one compound as disclosed herein may be administered, but in alternate embodiments, multiple compounds as disclosed herein may be administered.

As used herein, the term “effective amount” refers to an amount sufficient to achieve a desired result, including prevention or treatment of a disease. The term “effective amount” includes a “therapeutically effective amount.” The term “therapeutically effective amount” refers to an amount effective, at dosages and for periods of time necessary, to achieve the desired therapeutic result, such as reduction or inhibition of cell growth in the case of cancers. A therapeutically effective amount of the compounds as disclosed herein may vary according to factors such as the disease state, age, sex, and weight of the subject, and the ability of the disclosed compounds to elicit a desired response in the subject. Dosage regimens may be adjusted to provide the optimum therapeutic response. A therapeutically effective amount is also one in which any toxic or detrimental effects of the compounds as disclosed herein are reduced as compared with known compounds and are outweighed by the therapeutically beneficial effects.

As used herein, the term “tumor” or “cancer” refers to any abnormal proliferation of tissues, including solid and non-solid tumors. For instance, the composition and methods of the present disclosure can be utilized to treat cancers that manifest solid tumors such as breast cancer, colon cancer, gastric cancer, glioblastoma, head and neck cancer, kidney cancer, liver cancer, lung cancer, melanoma, ovarian cancer, pancreatic cancer, prostate cancer, and the like.

As used herein, the term “subject” refers mammals, non-mammals, and/or cells. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, fish and the like. The term “subject” does not denote a particular age or sex. Preferably, the subject is a human, particularly a human having cancer.

As used herein, the term “treat” or “treating” refers to the management and care of a subject for the purpose of combating the disease, condition, or disorder. Treating includes the administration of a compound or a pharmaceutical composition of the present disclosure to inhibit, ameliorate and/or improve the onset of the symptoms or complications, alleviating the symptoms or complications, or eliminating the disease, condition, or disorder.

As used herein, the term “administering” refers to any means for introducing the compounds and the pharmaceutical compositions as disclosed herein into the body, preferably into the systemic circulation. Examples include but are not limited to oral, buccal, sublingual, pulmonary, transdermal, transmucosal, as well as subcutaneous, intraperitoneal, intravenous, and intramuscular injection.

The compounds and pharmaceutical compositions utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

In some embodiments of the disclosed treatment methods, the subject may be administered a dose of a compound as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of a compound as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds may include doses falling within dose ranges having as endpoints any of these disclosed doses (e.g., 2.5 mg-200 mg).

In some embodiments, a minimal dose level of a compound for achieving therapy in the disclosed methods of treatment may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. In some embodiments, a maximal dose level of a compound for achieving therapy in the disclosed methods of treatment may not exceed about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. Minimal and/or maximal dose levels of the compounds for achieving therapy in the disclosed methods of treatment may include dose levels falling within ranges having as endpoints any of these disclosed dose levels (e.g., 500-2000 ng/kg body weight of the subject).

In some embodiments, the cancer to be treated is breast cancer, colon cancer, gastric cancer, glioblastoma, head and neck cancer, kidney cancer, liver cancer, lung cancer, melanoma, ovarian cancer, pancreatic cancer, prostate cancer, or a combination thereof.

In some embodiments, the method further comprises treating the subject with boron neutron capture therapy (BNCT) using the administered compound or composition, thereby the cancer is treated. Boron neutron capture therapy (BNCT) is an emerging treatment modality aimed at improving the therapeutic ratio for traditionally difficult to treat tumors. BNCT utilizes boronated agents to preferentially deliver boron-10 to tumors, which, after undergoing irradiation with neutrons, yields lithium-7 and an alpha particle. The alpha particle has a short range, therefore preferentially affecting tumor tissues while sparing more distal normal tissues. To date, BNCT has been studied clinically in a variety of disease sites, including glioblastoma multiforme, meningioma, head and neck cancers, lung cancers, breast cancers, hepatocellular carcinoma, sarcomas, cutaneous malignancies, extramammary Paget's disease, recurrent cancers, pediatric cancers, and metastatic disease.

In some embodiments, the compounds or pharmaceutical compositions as described herein is targeted to cancer cell mitochondria.

### Method of Reducing Reactive Oxygen and Nitrogen Species in Tumor Microenvironment of a Cancer

In another aspect, the present disclosure provides a method of reducing reactive oxygen and nitrogen species in tumor microenvironment of a cancer in a subject in need thereof. The method comprises administering to the subject an effective amount of the compound as disclosed herein, or the pharmaceutical composition as described herein.

“Reactive oxygen species” (i.e., ROS) refers to radicals, ions or molecules that have a single unpaired electron in their outermost shell of electrons. Due to this character, ROS are highly reactive. ROS can be categorized into two groups-free oxygen radicals and non-radical ROS. Free oxygen radicals include superoxide (O₂·⁻), hydroxyl radical (·OH), peroxyl radicals (ROO·), alkoxyl radicals (RO·), and thiyl peroxyl radicals (RSOO·). Non-radical ROS include hydrogen peroxide (H₂O₂), singlet oxygen (¹O₂), ozone/trioxygen (O₃), organic hydroperoxides (ROOH), hypochloride (HOCl), and highly reactive lipid- or carbohydrate-derived carbonyl compounds.

In some embodiments, the reactive oxygen species includes hydrogen peroxide and/or HOCl.

“Reactive nitrogen species” (i.e., RNS) refers to nitric oxide-derived compounds, including nitroxyl anion, nitrosonium cation, higher oxides of nitrogen, S-nitrosothiols, and dinitrosyl iron complexes. Examples include, but are not limited to, peroxynitrite (ONOO⁻), nitric oxide (NO·), nitrogen dioxide (·NO₂), dinitrogen dioxide (N₂O₂), and nitrosating species (N₂O₃).

In some embodiments, the reactive nitrogen species includes peroxynitrite (ONOO⁻).

The “tumor microenvironment” (TME) is a dynamic structure that is highly interactive with cancer cells and is shaped to a large extent by these cells. The composition of the TME varies between tumor types. Abnormal vasculature, hypoxia, acidic extracellular pH, an altered extracellular matrix, and stromal cells such as cancer-associated fibroblasts, macrophages, and immune cells are some of the characteristics of the TME. These characteristics contribute significantly to cancer progression.

In some embodiments, a NADPH oxidase (NOX) or an inducible nitric oxide synthase (iNOS) in the tumor microenvironment is inhibited or nitric oxide level in the tumor microenvironment is reduced, thereby reducing inflammation in the subject. In some embodiments, a NADPH oxidase (NOX) in the tumor microenvironment is inhibited. In some embodiments, an inducible nitric oxide synthase (iNOS) in the tumor microenvironment is inhibited. In some embodiments, nitric oxide level in the tumor microenvironment is reduced.

In some embodiments, nitric oxide level in the tumor microenvironment is reduced, and the nitric oxide comprises ONOO⁻.

### Method of Analyzing Reactive Oxygen or Nitrogen Species in Tumor Microenvironment of a Cancer

Another aspect of the disclosure provides a method of analyzing reactive oxygen or nitrogen species in tumor microenvironment of a cancer. The method comprises: introducing the compound or the pharmaceutical composition as described herein to the tumor microenvironment, whereby the reactive oxygen or nitrogen species reacts with the compound or composition to produce a product, and detecting the product thereby analyzing the reactive oxygen or nitrogen species in the tumor microenvironment.

In some embodiments, the reactive oxygen species includes hydrogen peroxide and/or HOCl. In some embodiments, the reactive nitrogen species includes peroxynitrite (ONOO⁻).

For example, proinflammatory oxidants such as peroxynitrite (ONOO), hypochlorous acid (HOCl), and H₂O₂ can undergo a nucleophilic addition reaction with phenylboronate moiety of the present compounds and produce a phenol product, which can be detected to analyze the reactive oxidant species. Suitable techniques for detecting and analyzing the product include use of radiolabels, mass spectrometry, and other known analytical methodologies.

### Method of Reducing or Inhibiting Cancer Cell Growth

Another aspect of the disclosure provides a method of reducing or inhibiting cancer cell growth in a subject in need thereof. The method comprises administering a therapeutically effective amount of the compound or the pharmaceutical composition as described herein to the subject.

In some embodiments, T cell killing of cancer cells is enhanced.

In some embodiments, the method further comprises treating the subject with boron neutron capture therapy (BNCT) using the administered compound or composition, thereby cancer cell growth is reduced or inhibited.

In some embodiments, the compound or composition is targeted to cancer cell mitochondria.

### Use

Another aspect of the disclosure provides a compound or a pharmaceutical composition for use in therapy. In some embodiments, the therapy is boron neutron capture therapy (BNCT).

Another aspect of the disclosure provides a compound or a pharmaceutical composition for use in treatment of cancer. In some embodiments, the cancer is breast cancer, colon cancer, gastric cancer, glioblastoma, head and neck cancer, kidney cancer, liver cancer, lung cancer, melanoma, ovarian cancer, pancreatic cancer, prostate cancer, or a combination thereof.

Another aspect of the disclosure provides a compound or a pharmaceutical composition for use in boron neutron capture therapy (BNCT) for cancer.

In some embodiments, the compound or composition for use as described herein (e.g., use in BNCT) is targeted to cancer cell mitochondria.

Another aspect of the disclosure provides a use of the compound as described herein for the manufacture of a medicament for the treatment of cancer.

### Kits

Another aspect of the disclosure provides a kit comprising a pharmaceutical composition comprising the compounds as disclosed herein and instructional material.

The term “instructional material” refers to a publication, a recording, a diagram, or any other medium of expression which is used to communicate the usefulness of the present pharmaceutical composition for one of the purposes set forth herein in a human. The instructional material can also, for example, describe an appropriate dose of the present pharmaceutical composition. The instructional material of the present kit can, for example, be affixed to a container which contains a pharmaceutical composition as disclosed herein or be shipped together with a container which contains the pharmaceutical composition. Alternatively, the instructional material can be shipped separately from the container with the intention that the instructional material and the pharmaceutical composition be used cooperatively by the recipient.

### EXAMPLES

The following examples are, of course, offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

In the following examples, polyphenolic boronates and mitochondria-targeted polyphenolic phytochemicals (e.g., magnolol [MGN] and honokiol [HNK]) were synthesized and developed. Their antiproliferative effects in brain cancer cells were tested. Results show that mitochondria-targeted (Mito) polyphenolic boronates (Mito-MGN-B and Mito-HNK-B) were slightly more potent than Mito-MGN and Mito-HNK in inhibiting proliferation of the U87MG cell line. Similar proliferation results also were observed in other cancer cell lines, such as MiaPaCa-2, A549 and UACC-62. Independent in vitro experiments indicated that reactive nitrogen species (e.g., peroxynitrite) and reactive oxygen species (e.g., hydrogen peroxide) stoichiometrically react with polyphenolic boronates and Mito-polphenolic boronates, forming polyphenols and Mito-polyphenols as major products. Previous reports suggest that both Mito-MGN and Mito-HNK activate cytotoxic T cells and inhibit immunosuppressive immune cells. It is hypothesized that Mito-polyphenolic boronate-based prodrugs may be used to inhibit tumor proliferation and mitigate oxidant formation in the tumor microenvironment, thereby generating Mito-polyphenols in situ, as well as showing activity in the tumor microenvironment.

The results of polyphenolic boronates in glioblastoma, lung cancer, melanoma, and pancreatic cancer cell lines were reported. The corresponding mitochondria-targeted polyphenols (mitochondria-targeted honokiol [Mito-HNK] and mitochondria-targeted magnolol [Mito-MGN]) have previously been tested in these cancer cells.

### Example 1. Synthesis of Polyphenolic Boronates

### Synthesis of HNK-B and HNK-B²

Honokiol boronate (HNK-B) and honokiol diboronate (HNK-B²) were prepared by reacting honokiol (HNK) in the presence of 2-(4-bromomethylphenyl)-4,4,5,5-tetramethyl-[1,3,2]-dioxaborolane and potassium carbonate in acetonitrile (MeCN).

To a mixture of HNK (0.5 g, 1.92 mmol) and anhydrous potassium carbonate (0.34 g, 2.4 mmol) in MeCN (5 mL) was added 2-(4-bromomethylphenyl)-4,4,5,5-tetramethyl-[1,3,2]-dioxaborolane (0.57 g, 1.90 mmol). The mixture was stirred at reflux overnight. Then, ethyl acetate was added to the mixture as well as water (H2O) (20 mL). The organic layer was washed twice with H₂O and dried over sodium sulfate (Na₂SO₄). The solvent was removed under reduced pressure. Purification by flash chromatography (pentane/diethyl ether [Et₂O] 9/1 and 8/2) delivered the corresponding HNK-B as a mixture of two isomers (0.4 g, 43% yield) and HNK-B² (0.1 g, 7%). High-resolution mass spectrometry (HRMS) calculated for HNK-B C₃₁H₃₅BO₄ [M+Na]⁺ 505.2526 returned 505.2526. HRMS calculated for HNK-B² C₄₄H₅₂B₂O₆ [M+Na]⁺ 721.3857 returned 721.3855.

### HNK-B

¹H NMR (400.13 MHz, CDCl₃) δ 7.83 (2H, 2d, J=7.8, 7.8), 7.50-7.33 (3H, m), 7.25-7.12 (2H, m), 7.05-6.82 (3H, m), 6.09-5.94 (2H, m), 5.17 (2H), 5.15-5.04 (5H, m), 3.48 (2H, 2d, J=6.6, 6.3), 3.39-3.35 (2H, m), 1.36-1.37 (12H, 2s). ¹³C NMR (75 MHz, CDCl₃) 156.1, 153.9, 153.2, 150.8, 140.5, 140.2, 137.8, 137.7, 136.5, 135.0, 134.9, 132.8, 132.1, 131.7, 131.2, 131.0, 130.7, 130.2, 130.1, 129.4, 129.0, 128.8, 127.9, 127.8, 126.3, 126.1, 124.7, 116.5, 115.9, 115.6, 115.5, 115.4, 113.5, 112.3, 83.9, 83.8, 70.6, 70.0, 39.4, 39.3, 35.2, 34.5, 29.7, 24.9.

### HNK-B²

¹H NMR (400.13 MHz, CDCl₃) δ 7.86 (2H, d, J=7.8), 7.78 (2H, d, J=7.8), 7.49 (2H, d, J=7.8), 7.44 (1H, d, J=1.8), 7.39-7.35 (3H, m), 7.15 (1H, d, J=1.9), 7.06 (1H, dd, J=8.3, 1.8), 6.93 (2H, dd, J=8.3, 2.9), 6.07-5.92 (2H, m), 5.15 (2H, s), 5.13-5.04 (4H, m), 5.07 (2H, s), 3.49 (2H, d, J=6.6), 3.37 (2H, d, J=6.6), 1.37 (12H, s), 1.35 (12H, s). ¹³C NMR (75 MHz, CDCl₃) δ 155.5, 153.9, 140.6, 140.5, 137.7, 136.9, 134.9, 134.8, 132.8, 131.3, 131.04, 131.02, 128.3, 128.3, 128.2, 127.8, 126.4, 126.1, 115.5, 113.4, 111.2, 83.8, 70.6, 69.9, 39.4, 34.6, 24.8.

A representative process of synthesis of HNK-B and HNK-B² is shown in Scheme 1.

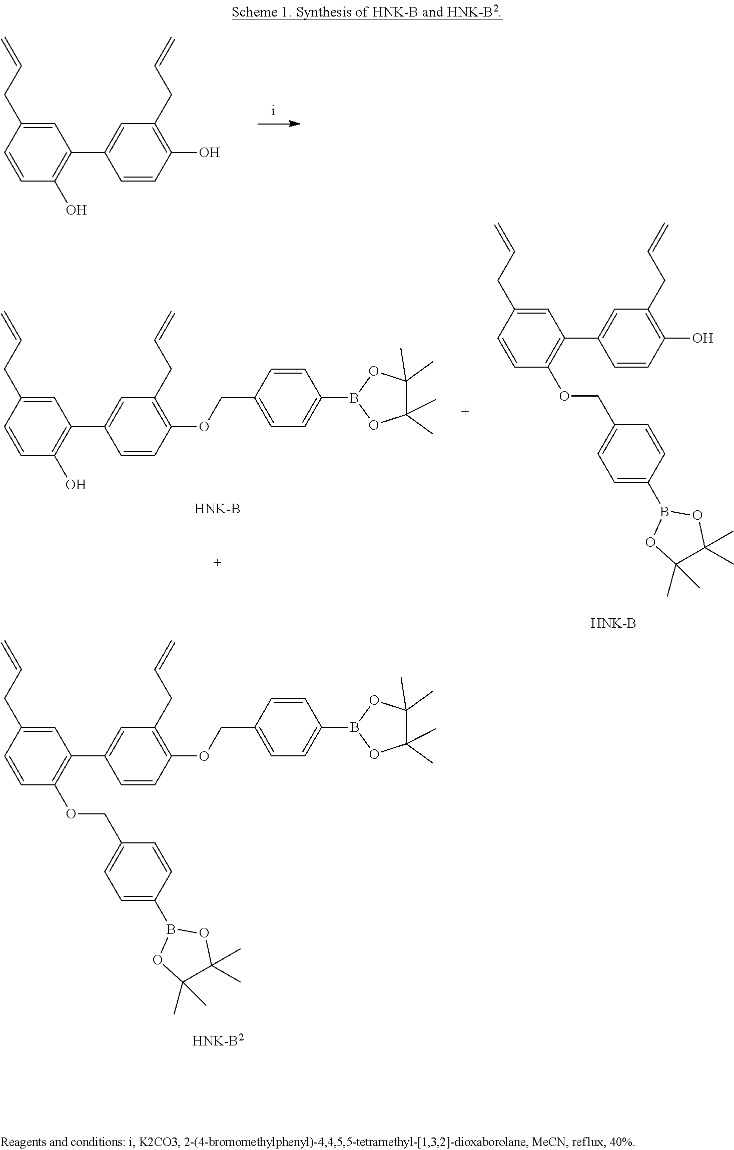

### Synthesis of Mito-HNK-B

Mitochondria-targeted honokiol boronate (Mito-HNK-B) was prepared by reacting Mito-HNK in the presence of 2-(4-bromomethylphenyl)-4,4,5,5-tetramethyl-[1,3,2]-dioxaborolane and potassium carbonate in MeCN.

To a mixture of Mito-HNK (0.36 g, 0.48 mmol) and anhydrous potassium carbonate (0.1 g, 0.7 mmol) in MeCN (5 mL) was added 2-(4-bromomethylphenyl)-4,4,5,5-tetramethyl-[1,3,2]-dioxaborolane (0.24 g, 0.7 mmol). The mixture was stirred at reflux overnight. Then, dichloromethane (CH₂Cl₂) was added to the reaction mixture as well as H₂O (20 mL). The organic layer was washed twice with H₂O and dried over Na₂SO₄. The solvent was removed under reduced pressure. The residue was washed with Et₂O. Purification by flash chromatography (from CH₂Cl₂ to CH₂Cl₂/ethanol [EtOH] 95/5) delivered the corresponding Mito-HNK-B as a mixture of two isomers as white solids (0.17 g, 37% yield). HRMS calculated for Mito-HNK-B C₅₉H₆₉BBrO₄P [M]⁺ 883.5031 returned 883.5028.

³¹P (400.13 MHz, CDCl₃) δ 24.49, 24.46. ¹H NMR (400.13 MHz, CDCl₃) δ 7.90-7.64 (17H, m), 7.47-7.30 (4H, m), 7.16-7.00 (2H, m), 6.94-6.83 (2H, m), 6.07-5.90 (2H, m), 5.15-4.95 (6H, m), 3.88 (1H, t, J=6.3), 3.90 (1H, t, J=6.3), 3.85-3.73 (2H, m), 3.50-3.31 (4H, m), 1.82-1.68 (3H, m), 1.67-1.55 (5H, m), 1.35-1.34 (12H, 2s), 1.30-1.17 (8H, m). ¹³C NMR (75 MHz, CDCl₃) δ 155.9, 155.3, 154.4, 153.9, 140.6, 137.8, 137.7, 137.1, 137.0, 134.94, 134.91, 134.89, 134.83, 133.7, 133.6, 132.7, 132.1, 131.3, 131.2, 131.1, 131.0, 130.8, 130.4, 130.3, 128.2, 128.0, 127.8, 127.7, 126.3, 126.1, 118.9, 118.0, 115.5, 115.4, 115.35, 115.32, 113.4, 112.7, 111.2, 110.7, 83.8, 83.7, 70.5, 69.9, 68.5, 67.9, 39.4, 34.6, 34.5, 30.4, 30.3, 29.7, 29.4, 29.3, 29.2, 29.1, 29.08, 26.1, 26.02, 24.9, 22.6 (d, J=49.8), 22.5 (J=4.4).

A representative process of synthesis of Mito-HNK-B is shown in Scheme 2.

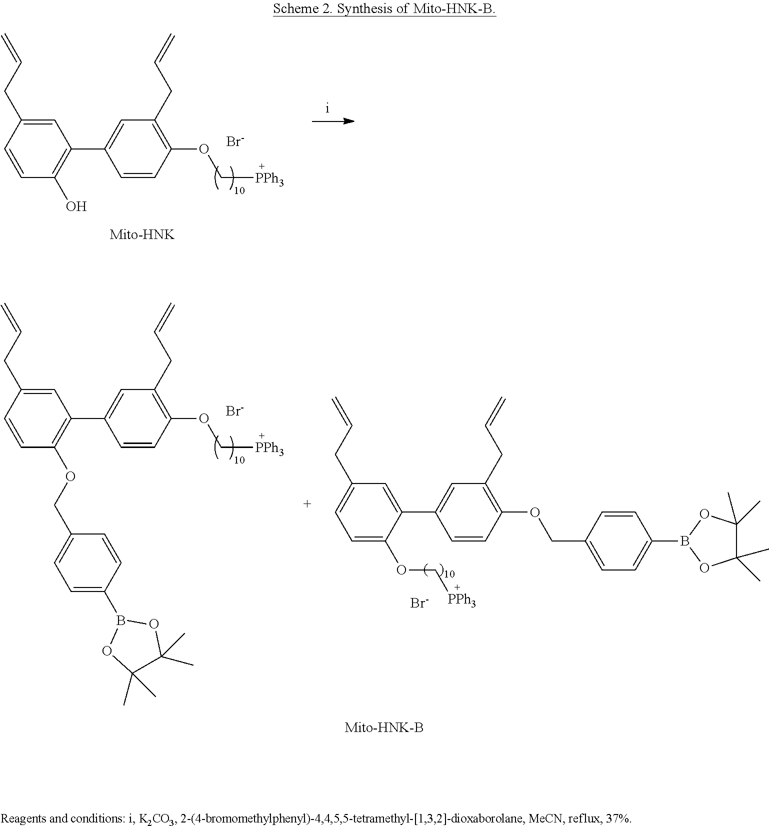

### Synthesis of MGN-B

Magnolol boronate (MGN-B) was prepared by reacting magnolol (MGN) in the presence of 2-(4-bromomethylphenyl)-4,4,5,5-tetramethyl-[1,3,2]-dioxaborolane and potassium carbonate in MeCN.

To a mixture of MGN (0.5 g, 1.92 mmol) and anhydrous potassium carbonate (0.34 g, 2.4 mmol) in MeCN (5 mL) was added 2-(4-bromomethylphenyl)-4,4,5,5-tetramethyl-[1,3,2]-dioxaborolane (0.57 g, 1.90 mmol). The mixture was stirred at reflux overnight. Then, ethyl acetate was added to the mixture as well as H₂O (20 mL). The organic layer was washed twice with H₂O and dried over Na₂SO₄. The solvent was removed under reduced pressure. Purification by flash chromatography (pentane/Et₂O 9/1 and 8/2) delivered the corresponding MGN-B (0.5 g, 55% yield). HRMS calculated for MGN-B C₃₁H₃₅BO₄ [M+Na]⁺ 505.2526 returned 505.2528.

¹H NMR (400.13 MHz, CDCl₃) δ 7.71 (2H, d, J=7.8), 7.21 (2H, d, J=7.9), 7.13-7.04 (3H, m), 7.02 (1H, d, J=1.6), 6.95 (1H, d, J=8.2), 6.91 (1H, d, J=8.2), 6.14 (1H, s), 6.01-5.87 (2H, m), 5.08-5.01 (6H, m), 3.33 (4H, d, J=3.3), 1.29 (12H, s). ¹³C NMR (75 MHz, CDCl₃) δ 153.2, 151.9, 139.2, 137.9, 137.3, 135.0, 133.4, 132.5, 132.2, 131.1, 129.3, 129.1, 128.1, 126.4, 126.1, 117.3, 115.9, 115.5, 114.5, 83.8, 71.8, 39.4, 24.8.

A representative process of synthesis of MGN-B is shown in Scheme 3.

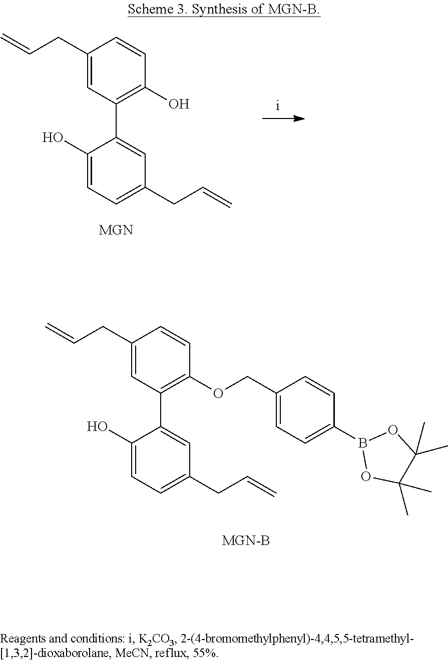 [SMILES: C=CCc1ccc(O)c(-c2cc(CC=C)ccc2O)c1 ; C=CCc1ccc(O)c(-c2cc(CC=C)ccc2OCc2ccc(B3OC(C)(C)C(C)(C)O3)cc2)c1]

### Synthesis of Mito-MGN-B

Mitochondria-targeted magnolol boronate (Mito-MGN-B) was prepared by reacting Mito-MGN in the presence of 2-(4-bromomethylphenyl)-4,4,5,5-tetramethyl-[1,3,2]-dioxaborolane and potassium carbonate in MeCN.

To a mixture of Mito-MGN (0.275 g, 0.37 mmol) and anhydrous potassium carbonate (0.076 g, 0.55 mmol) in MeCN (5 mL) was added 2-(4-bromomethylphenyl)-4,4,5,5-tetramethyl-[1,3,2]-dioxaborolane (0.14 g, 0.47 mmol). The mixture was stirred at reflux overnight. Then, CH₂Cl₂ was added to the reaction mixture as well as H₂O (20 mL). The organic layer was washed twice with H₂O and dried over Na₂SO₄. The solvent was removed under reduced pressure. The residue was washed with Et₂O. Purification by flash chromatography (from CH₂Cl₂ to CH₂Cl₂/EtOH 95/05) delivered the corresponding Mito-MGN-B as a white solid (0.15 g, 42% yield). HRMS calculated for Mito-MGN-B (C₅₉H₆₉BBrO₄P [M]⁺ 883.5031 returned 883.5032).

³¹P (400.13 MHz, CDCl₃) δ 24.32. ¹H NMR (400.13 MHz, CDCl₃) δ 7.85-7.64 (17H, m), 7.23-7.02 (6H, m), 6.87 (1H, d, J=5.5), 6.85 (1H, d, J=5.4), 6.01-5.89 (2H, m), 5.09-4.95 (6H, m), 3.81 (2H, t, J=6.5), 3.75-3.66 (2H, m), 3.37-3.29 (4H, m), 1.55-1.43 (3H, m), 1.32-1.29 (12H, s), 1.25-1.21 (7H, m), 1.16-1.05 (6H, m). ¹³C NMR (75 MHz, CDCl₃) δ 154.9, 154.4, 140.8, 137.85, 137.80, 134.95, 134.92, 134.62, 133.6, 133.5, 131.8, 131.79, 131.74, 131.4, 130.4, 130.3, 128.5, 128.2, 128.5, 127.9, 118.7, 117.9, 115.3, 112.8, 112.3, 83.7, 70.2, 68.5, 39.4, 30.4, 30.2, 29.3, 29.13, 29.10, 29.07, 28.9, 25.7, 24.9, 22.6 (d, J=49.2), 22.5 (d, J=4.4).

A representative process of synthesis of Mito-MGN-B is shown in Scheme 4.

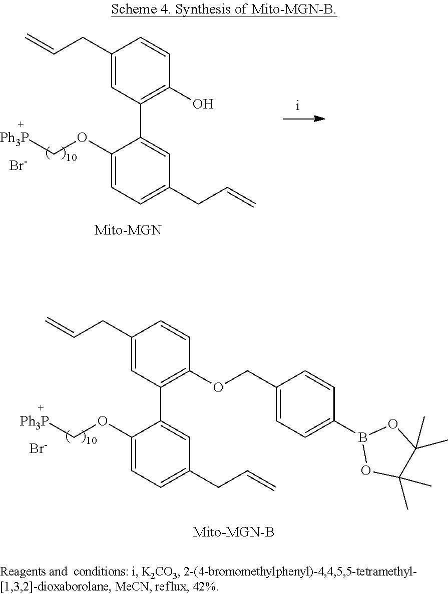

### Synthesis of MAG-BET

Magnolol ortho-boronate (MAG-BET) was prepared by reacting MGN in the presence of 2-(2-bromomethylphenyl)-4,4,5,5-tetramethyl-[1,3,2]-dioxaborolane and potassium carbonate in MeCN.

To a mixture of MGN (0.5 g, 1.92 mmol) and anhydrous potassium carbonate (0.34 g, 2.4 mmol) in MeCN (5 mL) was added 2-(2-bromomethylphenyl)-4,4,5,5-tetramethyl-[1,3,2]-dioxaborolane (0.57 g, 1.90 mmol). The mixture was stirred at reflux for 32 h. Then, ethyl acetate was added to the mixture as well as H₂O (20 mL). The organic layer was washed twice with H₂O and dried over Na₂SO₄. The solvent was removed under reduced pressure. Purification by flash chromatography (pentane/Et₂O 9/1 and 8/2) delivered the corresponding MAG-BET (0.25 g, 27% HRMS yield). calculated for MAG-BET C₃₁H₃₅BO₄ [M+Na]⁺ 505.2526 returned 505.2522.

¹H NMR (400.13 MHz, CDCl₃) δ 7.85 (1H, d, J=7.3), 7.41-7.28 (3H, m), 7.19-7.14 (2H, m), 7.11-7.04 (3H, m), 6.92 (1H, d, J=8.1), 6.46 (1H, s), 6.06-5.94 (2H, m), 5.41 (2H, s), 5.15-5.03 (4H, m), 3.41-3.37 (4H, m), 1.31 (12H, s). ¹³C NMR (75 MHz, CDCl₃) δ 153.5, 152.2, 142.4, 137.9, 137.5, 136.2, 133.7, 132.5, 132.0, 131.3, 131.2, 129.1, 128.9, 127.8, 127.6, 127.1, 126.5, 117.6, 115.7, 115.4, 113.8, 83.9, 71.1, 39.5, 39.4, 24.8.

A representative process of synthesis of MAG-BET is shown in Scheme 5.

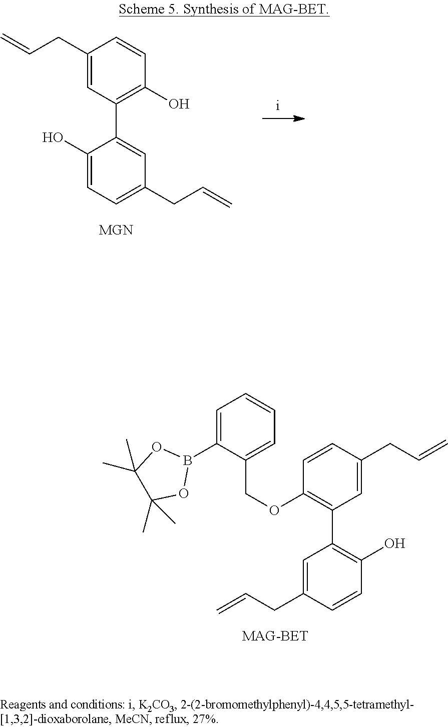 [SMILES: C=CCc1ccc(O)c(-c2cc(CC=C)ccc2O)c1 ; C=CCc1ccc(O)c(-c2cc(CC=C)ccc2OCc2ccccc2B2OC(C)(C)C(C)(C)O2)c1]

### Example 2. Polyphenolic Boronates Inhibit Tumor Cell Proliferation: Potential Mitigators of Oxidants in the Tumor Microenvironment

### Hydrophobicity of Polyphenolic Boronates

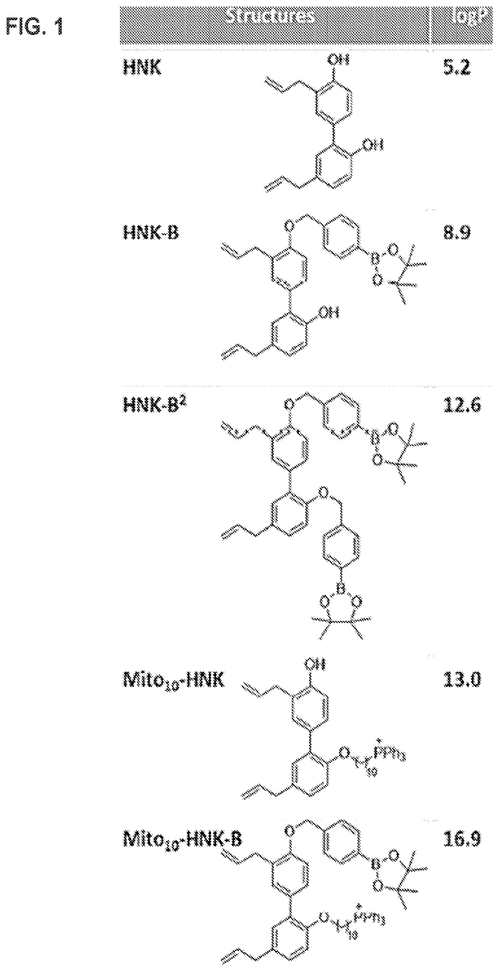

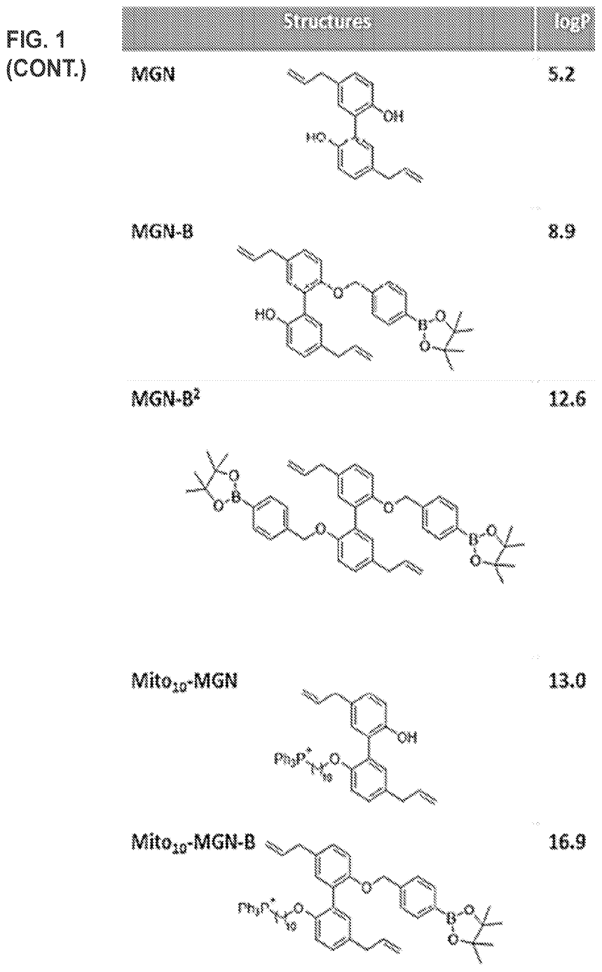

FIG. 1 lists the calculated partition coefficients for polyphenols (HNK, MGN, Mito-HNK, and Mito-MGN) and their boronate conjugates (HNK-B, HNK-B², Mito₁₀-HNK, Mito₁₀-HNK-B, MGN-B, MGN-B², Mito₁₀-MGN, Mito₁₀-MGN-B). Boronation increases the hydrophobicity of polyphenols. HNK-B² is significantly more hydrophobic than HNK.

### Antiproliferative Effects of Polyphenolic Boronates in Glioblastoma, Melanoma, Pancreatic Cancer, and Lung Cancer Cell Lines

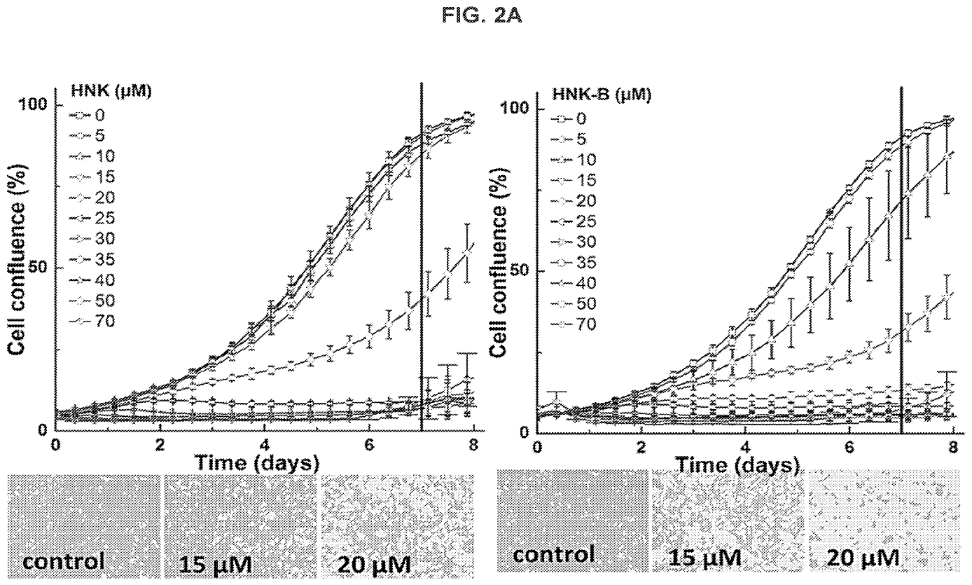

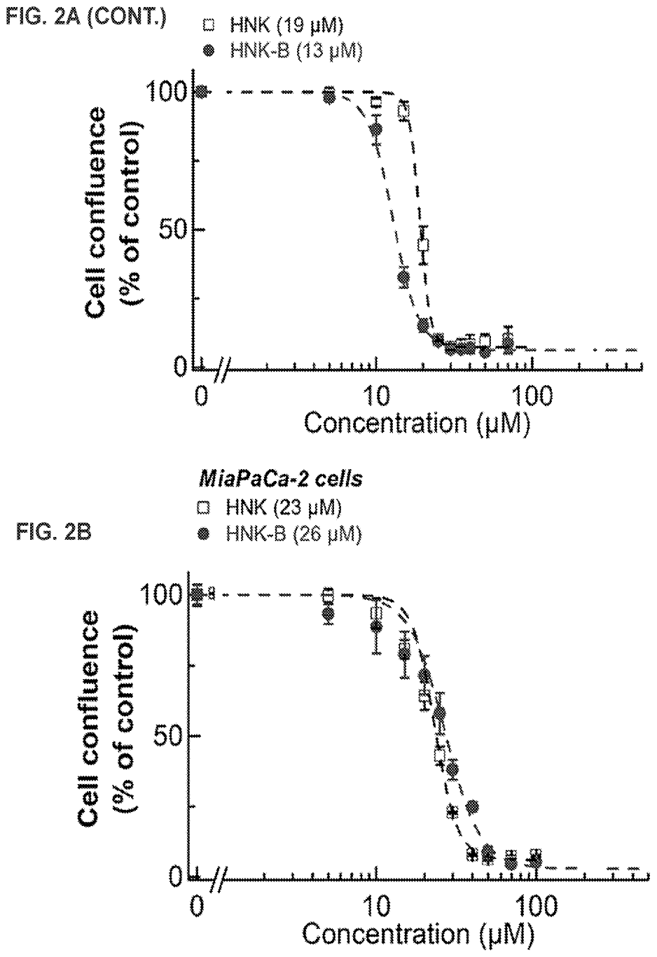

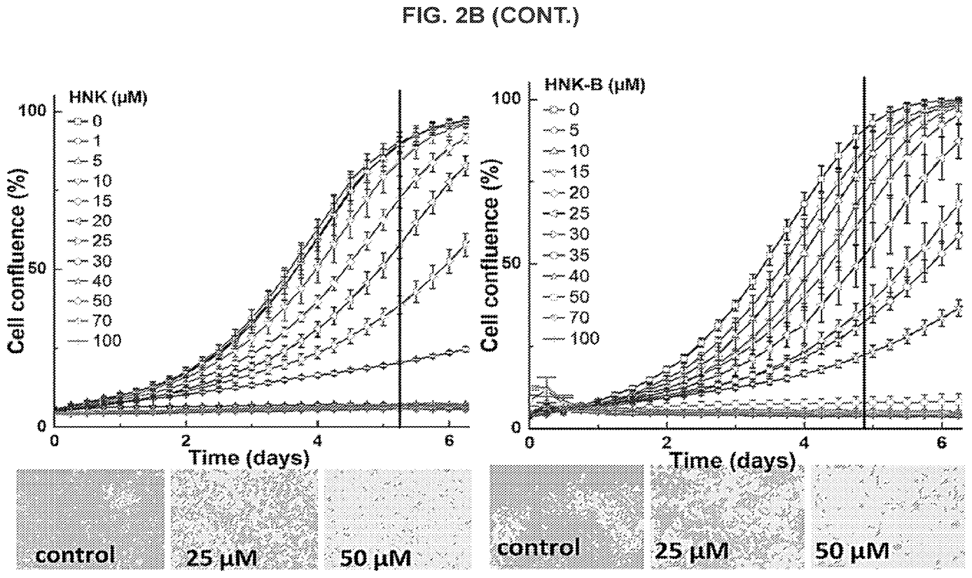

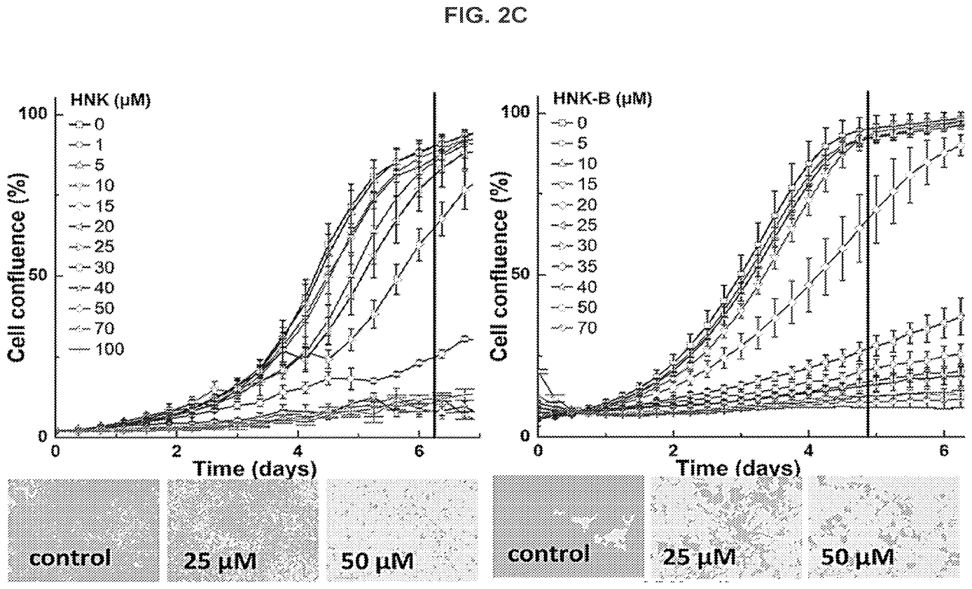

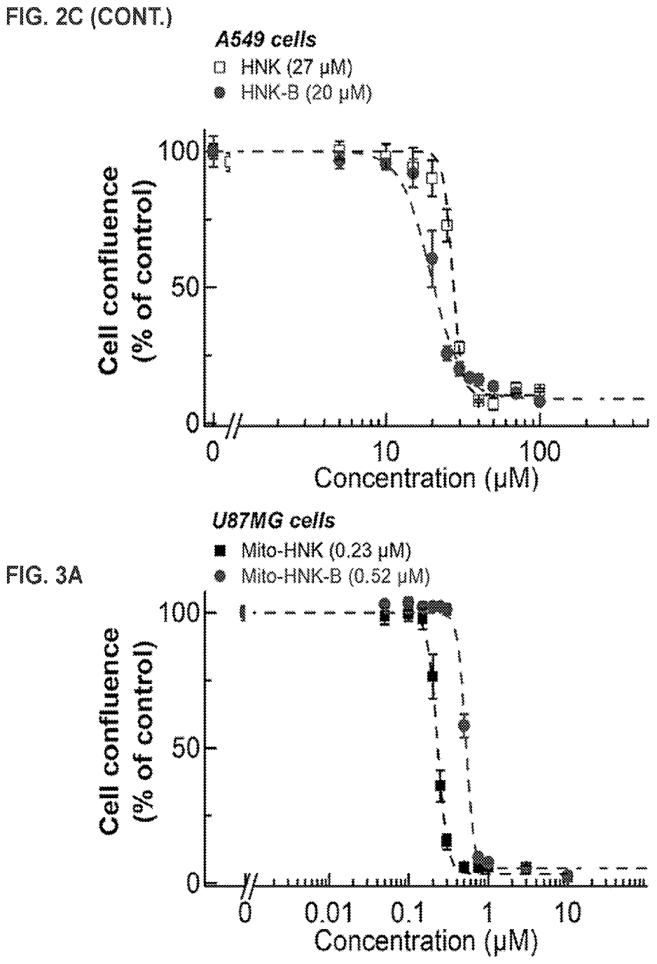

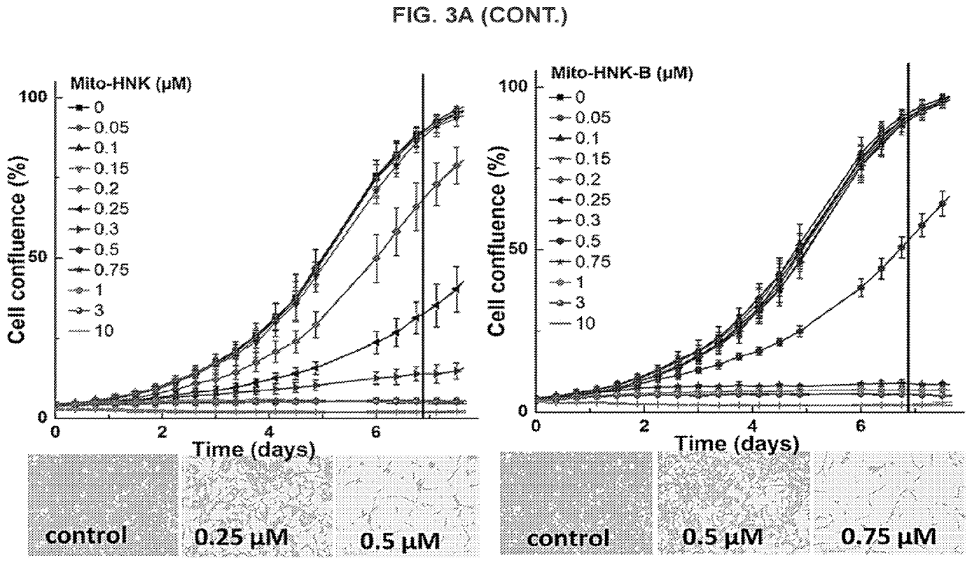

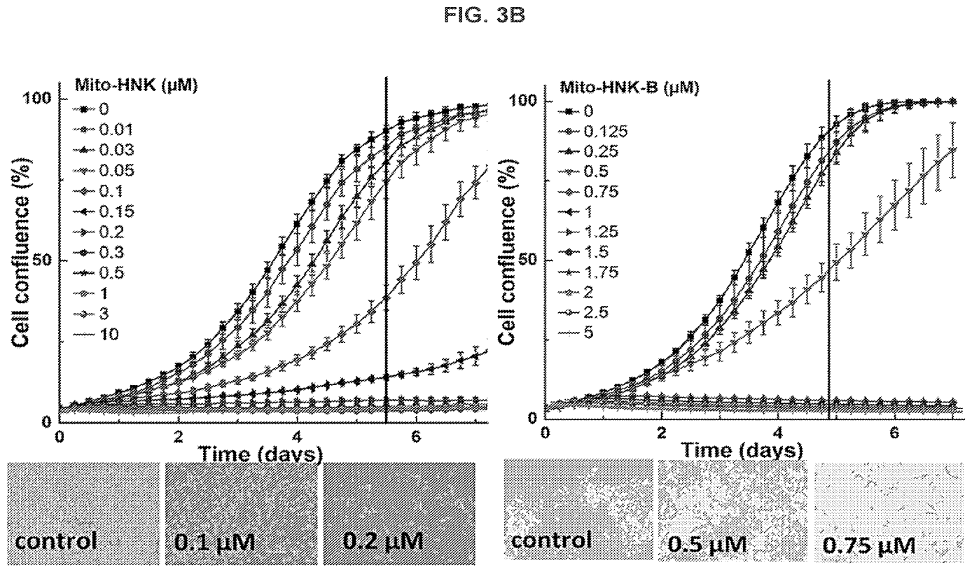

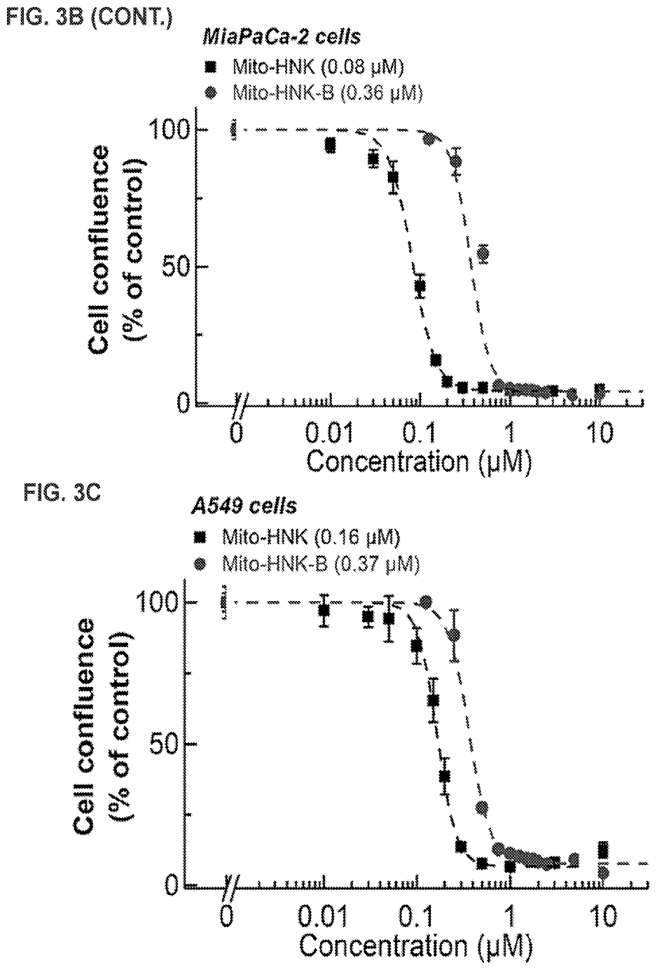

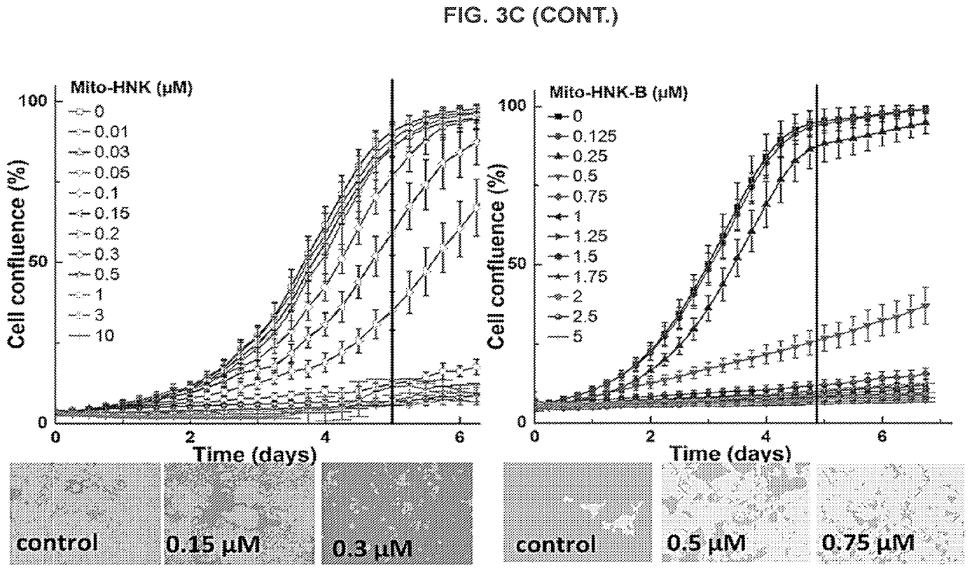

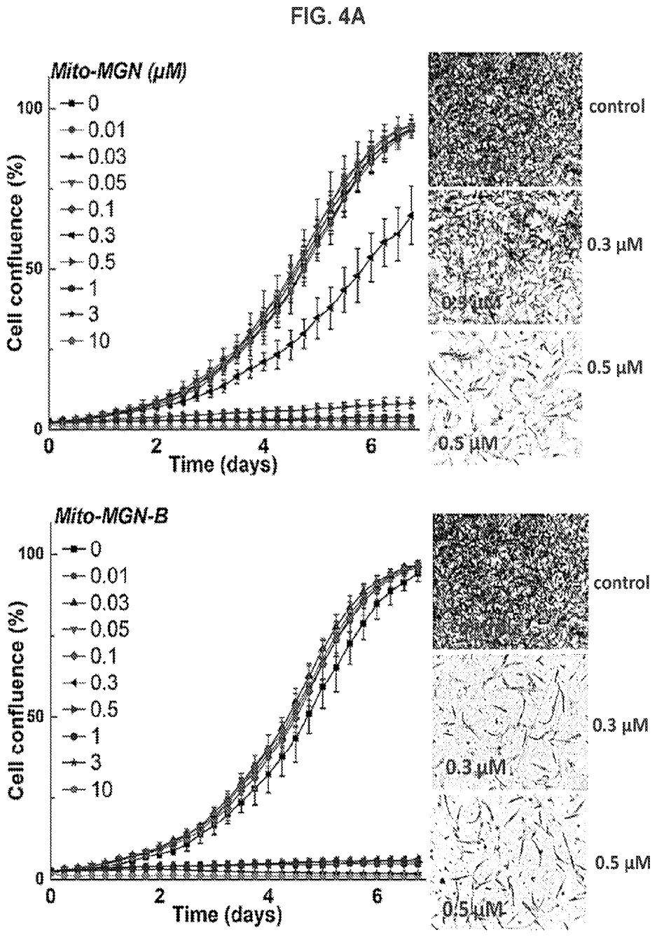

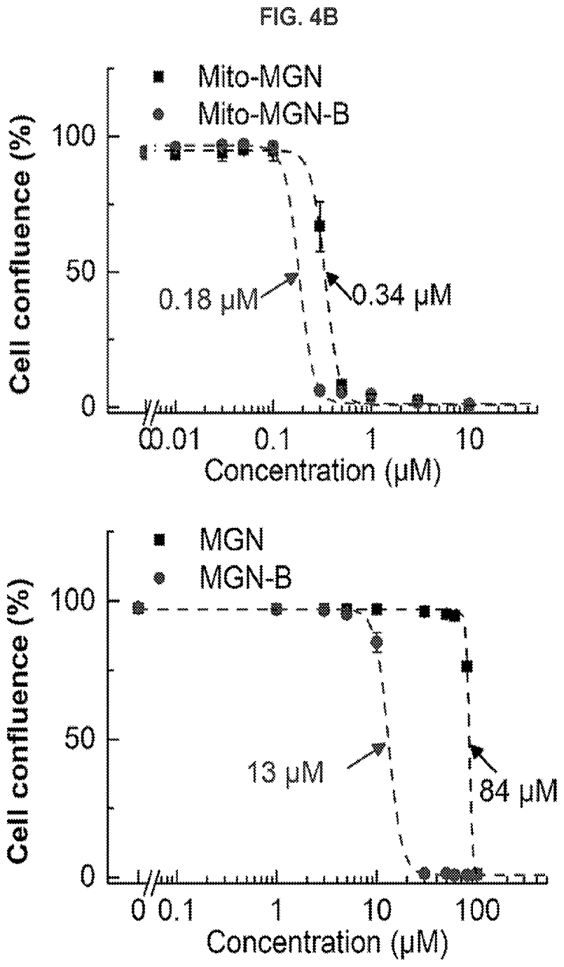

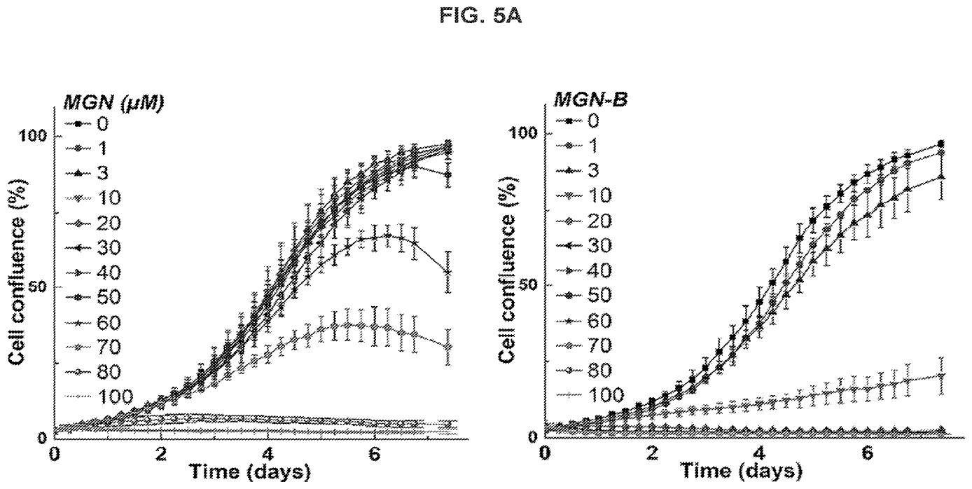

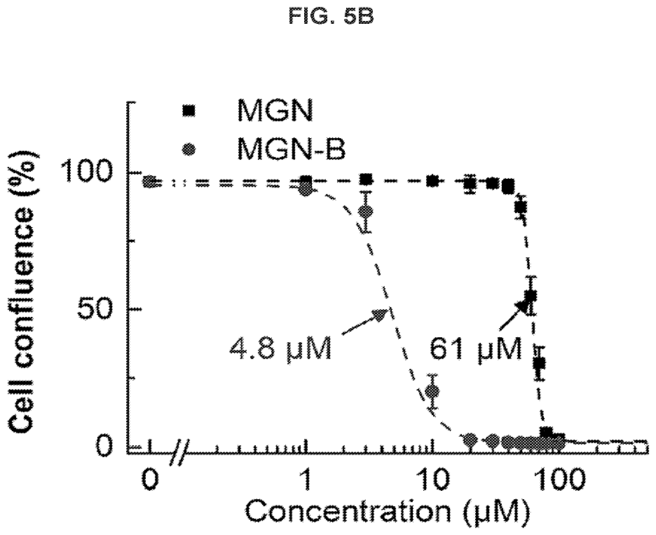

The effects of polyphenols (MGN, HNK) and their boronate analogs (Mito-MGN-B and Mito-HNK-B) in the glioblastoma (U87MG), melanoma (UACC-62), pancreatic cancer (MiaPaCa-2), and lung cancer (A549) cell lines (FIGS. 2A-2C, FIGS. 3A-3C, FIGS. 4A-4B and FIGS. 5A-5B) were compared. FIGS. 2A-2C show the dose-dependent effects of HNK and HNK-B in glioblastoma (U87MG), pancreatic cancer (MiaPaCa-2), and lung cancer (A549) cells. In these cell lines, the antiproliferative effects of HNK and HNK-B were nearly the same. FIGS. 3A-3C show the dose-dependent effects of Mito-HNK and Mito-HNK-B in these cell lines, and the antiproliferative effects of Mito-HNK and Mito-HNK-B were nearly the same. As shown in FIGS. 4A-4B, glioblastoma cells were treated with Mito-MGN/Mito-MGN-B and MGN/MGN-B. Boronation increased the potency of MGN-B as compared with MGN. Mito-MGN-B was also slightly more potent than Mito-MGN (FIGS. 4A-4B). In both cases, triphenylphosphonium (TPP⁺) conjugation (e.g., Mito-MGN and Mito-MGN-B) increased their antiproliferative potencies as compared with the parent compounds MGN/MGN-B. FIGS. 5A-5B show the dose-dependent effects of MGN and MGN-B in decreasing the proliferation of melanoma (UACC-62) cells.

### Reaction Between Polyphenolic Boronates and Oxidants

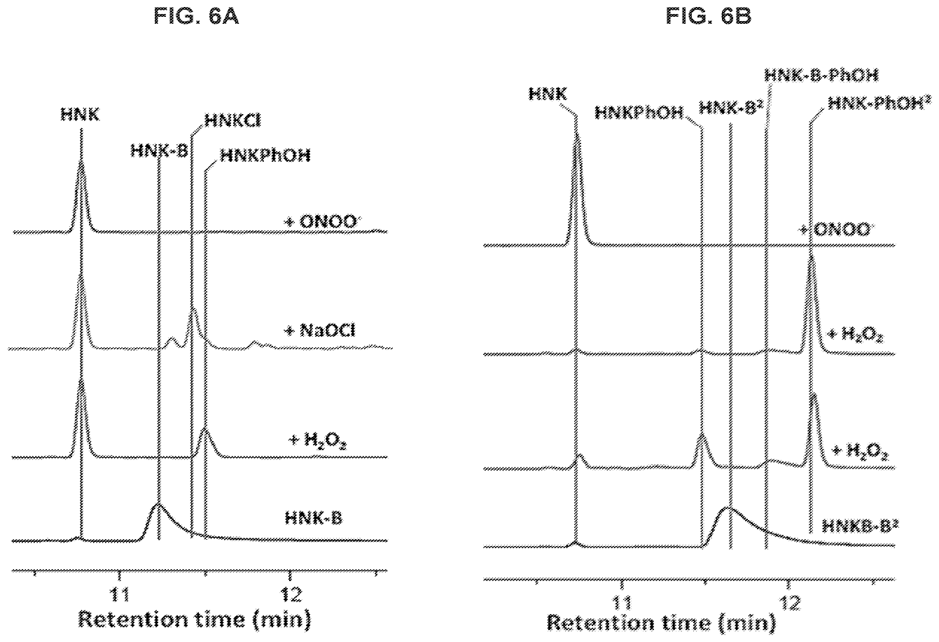

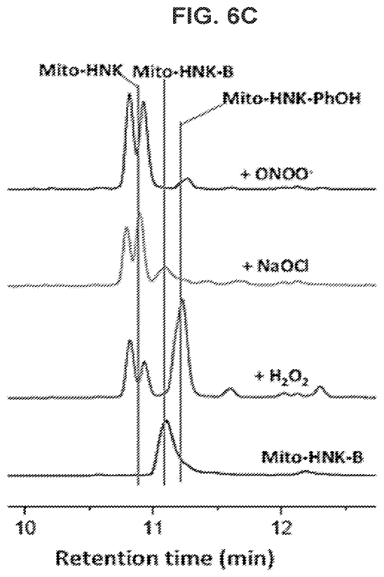

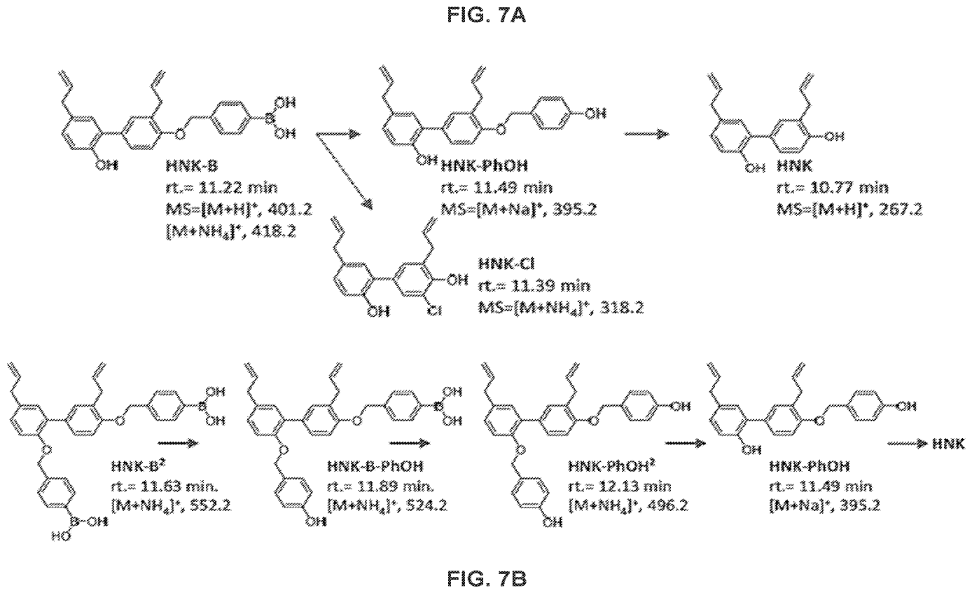

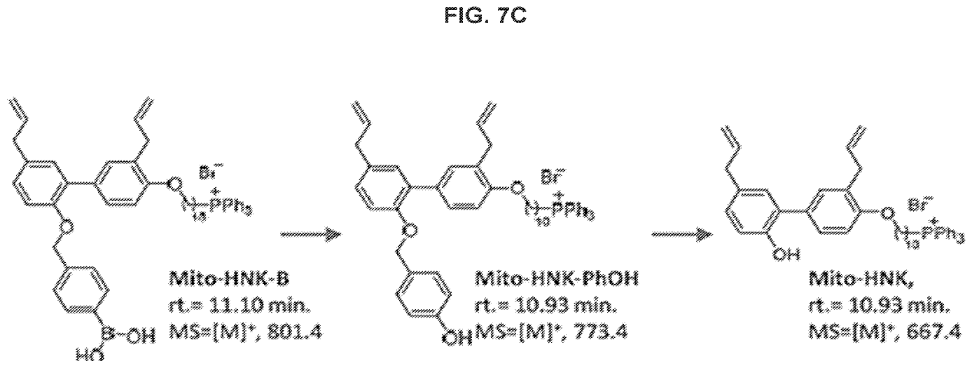

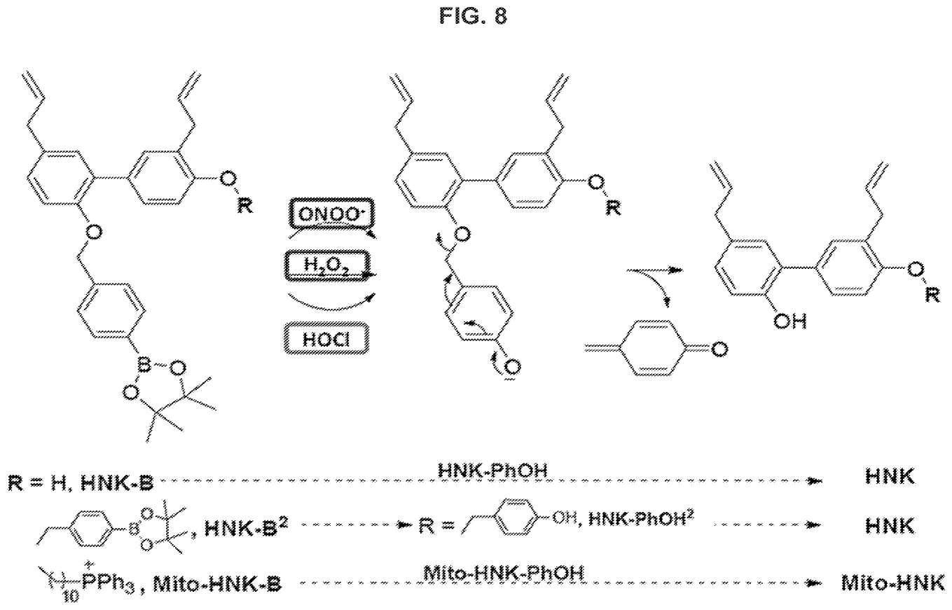

FIGS. 6A-6C and FIGS. 7A-7C show the HPLC traces and mass spectral parameters for HNK/HNK-B, Mito-HNK/Mito-HNK-B, and corresponding intermediates and products formed from the reaction with oxidants. In the presence of ONOO⁻, both HNK-B and Mito-HNK-B were rapidly oxidized to HNK and Mito-HNK. In contrast, in the presence of H₂O₂, smaller conversion of HNK-B to HNK (60%) and Mito-HNK-B to Mito-HNK (37%) was observed. However, at higher concentrations of H₂O₂, this conversion was increased. These results are consistent with the finding that ONOO⁻ reacts with boronates considerably faster than H₂O₂. Nitrated HNK and Mito-HNK were likely formed in minor quantities. HPLC results also show the formation of the hydroxy intermediate that triggers the self-immolative pathway (FIG. 8).

### Oxidative Cleavage of Polyphenolic Boronates and TPP⁺-Conjugated Polyphenolic Boronates

The polyphenolic boronate compounds consist of an arylboronic ester head group and a quinone methide group linked via a carbamate group to the parent polyphenolic compound (e.g., HNK or Mito-HNK). In the presence of oxidants such as H₂O₂, HOCl, and ONOO⁻, boronates are oxidized through insertion of an oxygen atom into the carbon-boron bond, forming the corresponding phenol intermediate. The phenoxide ion eliminates the quinone methide, releasing the polyphenolic compound via a decomposition process known as self-immolation (FIG. 8). As shown previously, ONOO⁻ reacts with boronates about one million times faster than H₂O₂ and one thousand times faster than HOCl. Phenols are the major product. Whereas the H₂O₂/boronate reaction is catalase-sensitive, the ONOO/boronate reaction is catalase-insensitive. Peroxy-caged luciferin (PCL-1) is oxidized by H₂O₂, HOCl, and ONOO to luciferin as a major product through a self-immolation mechanism. In an in vivo setting, PCL-1 has been used in luciferase-transfected mice xenografts to monitor oxidant formation through measurement of bioluminescence and in PET using a boronate-based positron emitting probe.

### Antitumor Activity of MTDs: Activation of Immune Cells

Mitochondria-targeted atovaquone (Mito-ATO) were showed to target both granulocytic-myeloid-derived suppressor cells (G-MDSCs) and regulatory T cells (T_{regs}) in the TME, resulting in significant decreases of both G-MDSCs and T_{regs} as determined by flow cytometry analysis. Intratumoral injection of Mito-ATO into primary tumors in a spontaneous tumor model triggered potent T cell immune responses locally and in distant tumor sites. Single-cell RNA sequencing revealed that Mito-ATO inhibits the expression of genes for OXPHOS and glycolysis in G-MDSCs and T_{regs} and facilitates the infiltration of CD4⁺ T cells in the tumor microenvironment. Other studies also revealed the immuno-modulatory and tumor-preventing effects of MTDs.

### Oxidants in the Tumor Microenvironment: Potential Inhibition by Polyphenolic Boronates

The TME changes dynamically. The TME consists of tumor cells, cancer-associated macrophages, lymphocytes, neutrophils, cancer-associated fibroblasts, endothelial cells, and vascular pericytes. The extracellular matrix in the TME consist of protein and polysaccharides. The dynamic interaction or the lack thereof between the tumor cells and the many cells and components in the TME regulates metabolism. MDSCs generate reactive oxygen and nitrogen species in the TME via activation of nicotinamide adenine dinucleotide phosphate oxidase (NOX) and inducible nitric oxide synthase (iNOS).

The anti-inflammatory effects of MTDs in Mito-Park mice were reported. MTDs such as Mito-apocynin decreased the expression of iNOS and NOX2, resulting in decreased oxidative and nitrative damage in neuronal cells. In another study, administration of an MTD (i.e., mitochondria-targeted carboxy-proxyl [Mito-CP]) inhibited cisplatin-induced renal toxicity by inhibiting pro-inflammatory mediators (iNOS in macrophages) in the kidney. Mitochondria-targeted compounds inhibit oxidants generated from NOX2. Inhibition of NOX4 (which presumably generates only H₂O₂) potentiates cancer immunotherapy. Recent clinical trials using iNOS inhibitors in breast cancer patients who were resistant to other forms of cancer therapies showed tumor shrinkage and enhanced patient survival. Inhibition of nitric oxide generated in the TME from tumor-associated macrophages decreased inflammatory mediators in the TME and enhanced killing of cancer cells by T cells.

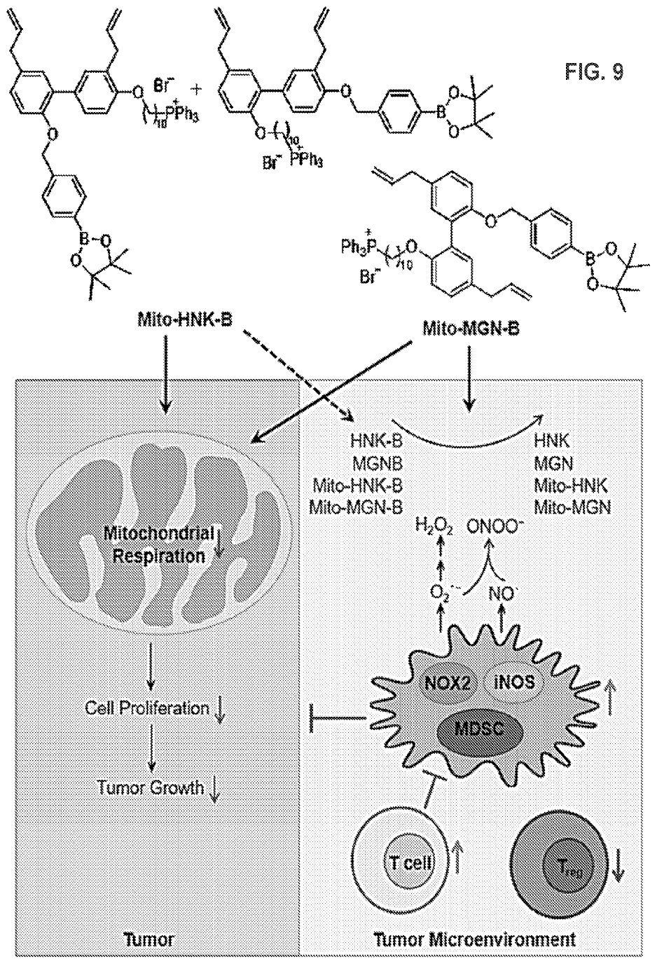

The reaction between polyphenolic boronates and reactive oxygen/nitrogen species released the parent polyphenolic compounds in situ. When MTD is released, it may inhibit iNOS and nitric oxide generation in the TME, thus decreasing inflammation. FIG. 9 shows the antioxidant potentials of Mito-HNK-B/Mito-MGN-B and HNK-B/MGN-B in the TME, especially the direct and nearly stoichiometric scavenging of ONOO⁻. The compound 3-(aminocarbonyl) furoxan-4-yl) methyl salicylate (AT38) was used to enhance the efficacy of immunotherapy in pancreatic cancer. AT38 inhibited the expression of arginase-1 and NOS-2 (or iNOS) in myeloid cells in the TME. AT38 mitigated chemokine nitration, promoting intratumoral infiltration of T cells. It is not clear if AT38 had any effect on NOX2 expression or on formation of superoxides and H₂O₂. Boronate-based drugs are likely to be more potent scavengers of ONOO⁻ (and HOCl), even in the presence of cellular reductants such as glutathione. Detection of the minor nitrated or chlorinated product from polyphenolic boronates can serve as a very reliable and diagnostic marker product of ONOO or HOCl generated in the TME. Preliminary results indicate that Mito-HNK inhibits oxidant formation from NOX2.

### Mitochondria-Targeted Boronates as Radiosensitizers

Boronated agents (e.g., boronophenylalanine) are used to enhance the therapeutic ratio in neutron treatment of cancers. This BNCT modality involves selective delivery of boron-10 to tumors followed by irradiation with neutrons. During this process, lithium-7 and an alpha particle that are generated destroy the tumor tissues. BNCT has been used clinically to treat cancers, including glioblastoma multiforme, head and neck cancers, lung cancer, and breast cancer. A critical requirement for enhanced therapeutic success is selective localization of boron-containing compounds in tumor tissues. Mitochondria-targeted boronates (Mito-HNK-B and Mito-MGN-B) that are selectively sequestered in tumor mitochondria could be more effective in capturing neutrons in mitochondria of tumors and enhance tumor killing.

Inhibition of mitochondrial respiration could alleviate tumor and TME hypoxia and act as radiation sensitizers. Mitochondria-targeted metformin and X-radiation sensitized the killing of pancreatic cancer cells. Clinical trials are ongoing with atovaquone and radiation therapy. Tumor hypoxia inhibits the efficacy of radiation and immunotherapies in cancer. Targeting mitochondrial metabolism in non-small cell lung cancer patients was reported to modify both the tumor and TME through a decrease in hypoxia and hypoxic gene expression. Mitochondria-targeted drugs decrease tumor hypoxia (or increase tumor oxygenation) by inhibiting mitochondrial respiration in tumors. Blood-brain-barrier-permeable aromatic boronates were effective in treating brain tumors using neutron capture therapy. Although it is not known whether mitochondria-targeted polyphenolic boronates (Mito-MGN-B and Mito-HNK-B) are blood-brain-barrier permeable, Mito-HNK inhibited metastasis of lung tumors to the brain in mice xenografts and showed blood-brain-barrier permeability.

### Mitochondria Metabolism and Racial Disparity in Cancer

Recent publications suggest that race and ethnicity are factors that affect mitochondrial metabolism in cancer cells, and that mitochondrial metabolism in white cancer patients is considerably different than in patients from other racial and ethnic groups. Studies have revealed distinct differences in the TME in Black cancer patients. In Black patients with bladder cancer, mitochondrial metabolism is considerably higher. Although more studies are needed to fully substantiate this trend, the differential metabolism provides a compelling rationale for testing mitochondria-targeted drugs in Black patients with bladder cancer.

The existence of racial and ethnic disparities has been identified in the breast cancer microenvironment. The TME of Black cancer patients with breast cancer exhibits increased level pro-tumorigenic factors (e.g., macrophages, T_{regs}, exhausted T cells) compared with white counterparts. Upregulation of OXPHOS genes was detected in tumor samples isolated from Black cancer patients. Tumors obtained from Black cancer patients have more mitochondria and PGC-la (proliferation-activated receptor gamma coactivator 1-alpha). Identification of biomarkers (e.g., hypoxia-induced genes) in these patients may provide additional insights. Black cancer patients were reported to exhibit a higher level of pro-inflammatory cytokines. Clinical trials targeting mitochondrial metabolism in cancer should include Black patients and patients from diverse racial and ethnic backgrounds. Presently, metformin is used as the sole drug of choice to test the mechanistic role of mitochondria in cancer disparity studies. Metformin is one of the most prescribed drugs for treating diabetes. Despite increased safety, its bioavailability is poor. Clinical trials revealed that Black cancer patients respond better to mitochondrial OXPHOS inhibitors (e.g., metformin) than white cancer patients. The present study opens the possibility of testing a new class of mitochondria-targeted drugs that target both the tumor mitochondria and TME to treat Black cancer patients.

The development of drugs that target both the tumor mitochondria and the TME would be a value-adding therapeutic advancement. Higher expression of OXPHOS in triple-negative breast cancer patients who received neoadjuvant chemotherapy was associated with worse treatment outcome. A novel class of mitochondria-targeted polyphenolic boronates were developed that inhibit tumor cell proliferation and scavenge oxidants such as H₂O₂, HOCl, and ONOO that are generated in the immunosuppressive TME. During this reaction, immunoactive parent polyphenols are regenerated. Specific polyphenols include HNK and MGN, which are active components of magnolia plant extract. Borono-L-phenylalanine was used to treat brain cancer in humans, so it is likely that polyphenolic boronates and the mitochondria-targeted boronate analogs are nontoxic and have high clinical and translational potential. The redox-based mitochondria-targeted polyphenolic boronates can be tested in the appropriate mice models (e.g., KPC mice) to assess their ability to inhibit reactive oxygen species/reactive nitrogen species-mediated nitration in the TME.

### Materials and Methods

The methods used in the synthesis, cell experiments, and statistical analysis follow standard scientific methods routinely used and described previously.

All chemicals and organic solvents were commercially available and were used as supplied. The reactions were monitored by thin layer chromatography (TLC) using silica gel Merck⁶⁰F254. Crude materials were purified by flash chromatography on Merck silica gel 60 (0.040-0.063 mm). ³¹P nuclear magnetic resonance (NMR), ¹H NMR, and ¹³C NMR spectra were recorded with spectrometers at 400.13 MHz and 75.54 MHz, respectively. ¹H NMR spectra were recorded at 400.13 MHz using a Bruker DPX AVANCE 400 spectrometer equipped with a quattro nucleus probe. ¹H NMR and ³¹P NMR were taken in deuterated chloroform (CDCl₃) using CDCl₃ and tetramethylsilane as internal references, respectively. Chemical shifts (8) are reported in ppm and J values in hertz.

### Cell Culture

The U87MG (ATCC Cat #HTB-14, human glioblastoma cancer cells), A549 (ATCC Cat #CCL-185, human lung cancer cells), and MiaPaCa-2 (ATCC Cat #CRL-1420, human pancreatic cancer cells) cell lines were purchased from the American Tissue Culture Collection (Manassas, VA, USA). The UACC-62 melanoma cell line was purchased from AddexBio (San Diego, CA; Cat #C0020003). All cell lines were regularly authenticated. All cell lines were grown at 37° C. in 5% carbon dioxide. The U87MG, A549, and UACC-62 cells were maintained in Roswell Park Memorial Institute Medium 1640 medium (Thermo Fisher Scientific, Waltham, MA, USA; Cat #11875) supplemented with 10% fetal bovine serum. MiaPaCa-2 cells were maintained in Dulbecco's Modified Eagle Medium (Thermo Fisher Scientific, Waltham, MA, USA; Cat #11965) and supplemented with 10% fetal bovine serum. All cells were stored in liquid nitrogen and used within 20 passages after thawing.

### Cell Proliferation

The IncuCyte Live-Cell Analysis System was used to continuously monitor cell proliferation, as described in earlier publications.

### Calculated Values of the Octanol/Water Partition Coefficients

The calculated octanol/H₂O partition coefficients (log P) of boronate analogs were assessed using a quantitative structure-activity relationship analysis and rational drug design as a measure of molecular hydrophobicity (FIG. 1). This method also uses a consensus model built using the ChemAxon software (San Diego, CA, USA).

### LC/MS Analysis

High-performance liquid chromatography (HPLC) analysis was performed using an Agilent 1200 apparatus equipped with ultraviolet-visible spectroscopy absorption and a mass spectrometry detector (single quadrupole).

In the studies on the reaction profile of ONOO oxidation of Mito-HNK-B and derivatives, 2 μL of sample was injected into the HPLC system equipped with a C18 column (Phenomenex, Kinetex Evo, 100×2.1 mm, 1.7 μm) equilibrated with 5% MeCN containing 0.1% (v/v) formic acid. The compounds were separated by a linear increase in MeCN phase concentration from 5% to 100% over 8 min and until 15 min at 100% MeCN using a flow rate of 0.21 mL/min. The peak areas detected by monitoring the absorption at 260 nm were used for the quantitation.

Oxidation of Boronates Derivatives by ONOO⁻, H₂O₂, and HOCl

The stock solutions of oxidants (HOCl and H₂O₂) were prepared freshly before each experiment; their concentrations were determined by spectrophotometry by reacting nitrite with H₂O₂, using the procedure described previously. ONOO⁻ was prepared according to the published procedure. Typically, ONOO⁻ was synthesized in a reaction of 0.6 M nitrite with 0.7 M H₂O₂ at pH 13. Excess H₂O₂ was removed by passage through a column of MnO₂ and the solution was frozen at −20° C. The liquid over the frozen solid was collected and stored at −80° C. Immediately prior to each experiment, the concentration of ONOO was determined spectrally at 302 nm (ε=1.7× 103 M⁻¹ cm⁻¹) after dilution in 0.1 M sodium hydroxide to ~10 mM concentration.

Stock solutions of HNK-B, HNK-B², and Mito-HNK-B were prepared in dimethyl sulfoxide at a 10 mM concentration and this solution was added directly to the buffer to obtain the desired concentration. HPLC analysis indicated that HNK-B, HNK-B², and Mito-HNK-B (pinacolate ester) undergo fast hydrolysis to the corresponding boronic acid formed upon dilution in the aqueous phosphate buffer. Thus, the boronic acid species were tested with the oxidants. When studying HNK-B and Mito-HNK-B oxidations by HOCl, stock solutions (10 mM) in MeCN were added to the phosphate buffer (100 mM, pH 7.4) to obtain a final concentration of the probe of 500 μM. Dimethyl sulfoxide solvent was avoided due to known rapid quenching of HOCl by dimethyl sulfoxide.

A study of the reactivity of HNK-B² in the presence of sodium hypochlorite (NaOCl) was not possible because its solubility is too low in MeCN and water.

### Abbreviations

AT38 3-(aminocarbonyl) furoxan-4-yl) methyl salicylate
BNC boron neutron capture therapy
CDCl3 deuterated chloroform
CH2Cl2 dichloromethane
Et2O diethyl ether
EtOH ethanol
G-MDSCs granulocytic-myeloid-derived suppressor cells
H2O water
H2O2 hydrogen peroxide
HNK honokiol
HNK-B honokiol boronate
HNK-B2 honokiol diboronate
HOCl hypochlorous acid
HPLC high-performance liquid chromatography
HRMS high-resolution mass spectrometry
IC50 half maximal inhibitory concentration
iNOS inducible nitric oxide synthase
log P octanol/H2O partition coefficients
MAG-BET magnolol ortho-boronate
MeCN acetonitrile
MGN magnolol
MGN-B magnolol boronate
Mito mitochondria-targeted
Mito-ATO mitochondria-targeted atovaquone
Mito-CP mitochondria-targeted carboxy-proxyl
Mito-HNK mitochondria-targeted honokiol
Mito-HNK-B mitochondria-targeted honokiol boronate
Mito-MGN mitochondria-targeted magnolol
Mito-MGN-B mitochondria-targeted magnolol boronate
MTD mitochondria-targeted drug
NOX nicotinamide adenine dinucleotide phosphate oxidase
Na2SO4 sodium sulfate
NaOCl sodium hypochlorite
NMR nuclear magnetic resonance
ONOO− peroxynitrite
OXPHOS oxidative phosphorylation
PET positron-emission tomography
SD standard deviation
Tregs regulatory T cells
TPP+ triphenylphosphonium
TLC thin layer chromatography
TME tumor microenvironment
δ chemical shifts

### REFERENCES

1. Malouff, T. D., Seneviratne, D. S., Ebner, D. K., Stross, W. C., Waddle, M. R., Trifiletti, D. M., and Krishnan, S. (2021) Boron Neutron Capture Therapy: A Review of Clinical Applications. Front Oncol 11, 601820
2. Luderer, M. (2019) Development of Novel Tumor-Targeted Compounds for Boron Neutron Capture Therapy. PhD, Washington University in St. Louis
3. Chen, Y. W., Chou, F. I., Huang, W. S., Lin, K. H., Pan, P. S., Kuo, Y. C., Hsu, S. M., Chen, J. K., Wang, C. W., Chen, K. H., Lan, T. L., and Lee, J. C. (2019) Boron Neutron Capture Therapy: A New Generation of Targeted Charged-Particle Radiotherapy. Journal of Scientific & Technical Research 13, 9687-9689
4. Turkez, H., Arslan, M. E., Tatar, A., and Mardinoglu, A. (2021) Promising potential of boron compounds against Glioblastoma: In Vitro antioxidant, anti-inflammatory and anticancer studies. Neurochem. Int. 149, 105137
5. Chio, C. M., Huang, Y. C., Chou, Y. C., Hsu, F. C., Lai, Y. B., and Yu, C. S. (2020) Boron Accumulation in Brain Tumor Cells through Boc-Protected Tryptophan as a Carrier for Boron Neutron Capture Therapy. ACS Med Chem Lett 11, 589-596
6. Maslah, H., Pethe, S., and Labruère, R. (2021) Boronic acid/boronate prodrugs for cancer treatment: current status and perspectives. Future Med Chem 13, 859-861
7. Meiyanto, E., R. A. S., R. I. J., R. Y. U., and, D. N., Febri Wulandari, Mitsunori Kirihata. (2020) Synthesis of new boron containing compound (CCB-2) based on curcumin structure and its cytotoxic effect against cancer cells, Issue: 2
8. Canturk, Z., Tunali, Y., Korkmaz, S., and Gulbaş, Z. (2016) Cytotoxic and apoptotic effects of boron compounds on leukemia cell line. Cytotechnology 68, 87-93
9. Koldemir-Gündüz, M., Aydin, H. E., Berikten, D., Kaymak, G., Köse, D. A., and Arslantaş, A. (2021) Synthesis of New Boron Derived Compounds; Anticancer, Antioxidant and Antimicrobial Effect in Vitro Glioblastoma Tumor Model. J Korean Neurosurg Soc 64, 864-872
10. Song, S., Gao, P., Sun, L., Kang, D., Kongsted, J., Poongavanam, V., Zhan, P., and Liu, X. (2021) Recent developments in the medicinal chemistry of single boron atom-containing compounds. Acta Pharm Sin B 11, 3035-3059
11. Franzyk, H., and Christensen, S. B. (2021) Targeting Toxins toward Tumors. Molecules 26
12. Kahraman, E., and Göker, E. (2022) Boric acid exert anti-cancer effect in poorly differentiated hepatocellular carcinoma cells via inhibition of AKT signaling pathway. J. Trace Elem. Med Biol. 73, 127043
13. Sikora, A., Zielonka, J., Dębowska, K., Michalski, R., Smulik-Izydorczyk, R., Pięta, J., Podsiadly, R., Artelska, A., Pierzchala, K., and Kalyanaraman, B. (2020) Boronate-Based Probes for Biological Oxidants: A Novel Class of Molecular Tools for Redox Biology. Front Chem 8, 580899
14. Grzelakowska, A., Modrzejewska, J., Kolińska, J., Szala, M., Zielonka, M., Dębowska, K., Zaklos-Szyda, M., Sikora, A., Zielonka, J., and Podsiadly, R. (2022) Water-soluble cationic boronate probe based on coumarin imidazolium scaffold: Synthesis, characterization, and application to cellular peroxynitrite detection. Free Radic Biol Med 179, 34-46
15. Zielonka, J., Podsiadly, R., Zielonka, M., Hardy, M., and Kalyanaraman, B. (2016) On the use of peroxy-caged luciferin (PCL-1) probe for bioluminescent detection of inflammatory oxidants in vitro and in vivo-Identification of reaction intermediates and oxidant-specific minor products. Free Radical Biol. Med. 99, 32-42
16. Cheng, G., Pan, J., Podsiadly, R., Zielonka, J., Garces, A. M., Dias Duarte Machado, L. G., Bennett, B., McAllister, D., Dwinell, M. B., You, M., and Kalyanaraman, B. (2020) Increased formation of reactive oxygen species during tumor growth: Ex vivo low-temperature EPR and in vivo bioluminescence analyses. Free Radic Biol Med 147, 167-174
17. Kuivila, H. G. (1954) Electrophilic Displacement Reactions. III.

Kinetics of the Reaction between Hydrogen Peroxide and Benzeneboronic Acid1. Journal of the American Chemical Society 76, 870-874
18. Sikora, A., Zielonka, J., Lopez, M., Dybala-Defratyka, A., Joseph, J., Marcinek, A., and Kalyanaraman, B. (2011) Reaction between peroxynitrite and boronates: EPR spin-trapping, HPLC Analyses, and quantum mechanical study of the free radical pathway. Chem. Res. Toxicol. 24, 687-697
19. Sikora, A., Zielonka, J., Adamus, J., Debski, D., Dybala-Defratyka, A., Michalowski, B., Joseph, J., Hartley, R. C., Murphy, M. P., and Kalyanaraman, B. (2013) Reaction between peroxynitrite and triphenylphosphonium-substituted arylboronic acid isomers: identification of diagnostic marker products and biological implications. Chem. Res. Toxicol. 26, 856-867
20. Van De Bittner, G. C., Dubikovskaya, E. A., Bertozzi, C. R., and Chang, C. J. (2010) In vivo imaging of hydrogen peroxide production in a murine tumor model with a chemoselective bioluminescent reporter. Proceedings of the National Academy of Sciences of the United States of America 107, 21316-21321
21. Carroll, V., Michel, B. W., Blecha, J., VanBrocklin, H., Keshari, K., Wilson, D., and Chang, C. J. (2014) A Boronate-Caged [18F]FLT Probe for Hydrogen Peroxide Detection Using Positron Emission Tomography. Journal of the American Chemical Society 136, 14742-14745
22. Lippert, A. R., Van de Bittner, G. C., and Chang, C. J. (2011) Boronate oxidation as a bioorthogonal reaction approach for studying the chemistry of hydrogen peroxide in living systems. Acc. Chem. Res. 44, 793-804
23. Zielonka, J., Zielonka, M., Sikora, A., Adamus, J., Joseph, J., Hardy, M., Ouari, O., Dranka, B. P., and Kalyanaraman, B. (2012) Global profiling of reactive oxygen and nitrogen species in biological systems: high-throughput real-time analyses. J. Biol. Chem. 287, 2984-2995
24. Zielonka, J., Sikora, A., Hardy, M., Joseph, J., Dranka, B. P., and Kalyanaraman, B. (2012) Boronate probes as diagnostic tools for real time monitoring of peroxynitrite and hydroperoxides. Chem. Res. Toxicol. 25, 1793-1799
25. Zielonka, J., Sikora, A., Joseph, J., and Kalyanaraman, B. (2010) Peroxynitrite is the major species formed from different flux ratios of co-generated nitric oxide and superoxide: direct reaction with boronate-based fluorescent probe. J. Biol. Chem. 285, 14210-14216
26. Rios, N., Radi, R., Kalyanaraman, B., and Zielonka, J. (2020) Tracking isotopically labeled oxidants using boronate-based redox probes. J. Biol. Chem. 295, 6665-6676
27. Sieracki, N. A., Gantner, B. N., Mao, M., Horner, J. H., Ye, R. D., Malik, A. B., Newcomb, M. E., and Bonini, M. G. (2013) Bioluminescent detection of peroxynitrite with a boronic acid-caged luciferin. Free Radic Biol Med 61, 40-50
28. Kalyanaraman, B., Hardy, M., Podsiadly, R., Cheng, G., and Zielonka, J. (2017) Recent developments in detection of superoxide radical anion and hydrogen peroxide: Opportunities, challenges, and implications in redox signaling. Archives of Biochemistry and Biophysics 617, 38-47
29. Maslah, H., Skarbek, C., Pethe, S., and Labruère, R. (2020) Anticancer boron-containing prodrugs responsive to oxidative stress from the tumor microenvironment. Eur. J. Med. Chem. 207, 112670
30. Jia, P., Dai, C., Cao, P., Sun, D., Ouyang, R., and Miao, Y. (2020) The role of reactive oxygen species in tumor treatment. RSC Adv 10, 7740-7750
31. Wang, T., and Xu, H. (2022) Multi-faced roles of reactive oxygen species in anti-tumor T cell immune responses and combination immunotherapy. Exploration of Medicine 3, 77-98
32. Liu, R., Peng, L., Zhou, L., Huang, Z., Zhou, C., and Huang, C. (2022) Oxidative Stress in Cancer Immunotherapy: Molecular Mechanisms and Potential Applications. Antioxidants 11, 853
33. Kotsafti, A., Scarpa, M., Castagliuolo, I., and Scarpa, M. (2020) Reactive Oxygen Species and Antitumor Immunity-From Surveillance to Evasion. Cancers (Basel) 12
34. Lian, X., Yang, K., Li, R., Li, M., Zuo, J., Zheng, B., Wang, W., Wang, P., and Zhou, S. (2022) Immunometabolic rewiring in tumorigenesis and anti-tumor immunotherapy. Mol Cancer 21, 27
35. Aboelella, N. S., Brandle, C., Kim, T., Ding, Z. C., and Zhou, G. (2021) Oxidative Stress in the Tumor Microenvironment and Its Relevance to Cancer Immunotherapy. Cancers (Basel) 13
36. Gabrilovich, D. I., and Nagaraj, S. (2009) Myeloid-derived suppressor cells as regulators of the immune system. Nat. Rev. Immunol. 9, 162-174
37. Krishnamoorthy, M., Gerhardt, L., and Maleki Vareki, S. (2021) Immunosuppressive Effects of Myeloid-Derived Suppressor Cells in Cancer and Immunotherapy. Cells 10
38. Lu, T., Ramakrishnan, R., Altiok, S., Youn, J. I., Cheng, P., Celis, E., Pisarev, V., Sherman, S., Sporn, M. B., and Gabrilovich, D. (2011) Tumor-infiltrating myeloid cells induce tumor cell resistance to cytotoxic T cells in mice. J. Clin. Invest. 121, 4015-4029
39. Buck, M. D., O'Sullivan, D., and Pearce, E. L. (2015) T cell metabolism drives immunity. J. Exp. Med. 212, 1345-1360
40. De Sanctis, F., Lamolinara, A., Boschi, F., Musiu, C., Caligola, S., Trovato, R., Fiore, A., Frusteri, C., Anselmi, C., Poffe, O., Cestari, T., Canè, S., Sartoris, S., Giugno, R., Del Rosario, G., Zappacosta, B., Del Pizzo, F., Fassan, M., Dugnani, E., Piemonti, L., Bottani, E., Decimo, I., Paiella, S., Salvia, R., Lawlor, R. T., Corbo, V., Park, Y., Tuveson, D. A., Bassi, C., Scarpa, A., Iezzi, M., Ugel, S., and Bronte, V. (2022) Interrupting the nitrosative stress fuels tumor-specific cytotoxic T lymphocytes in pancreatic cancer. J Immunother Cancer 10
41. Molon, B., Ugel, S., Del Pozzo, F., Soldani, C., Zilio, S., Avella, D., De Palma, A., Mauri, P., Monegal, A., Rescigno, M., Savino, B., Colombo, P., Jonjic, N., Pecanic, S., Lazzarato, L., Fruttero, R., Gasco, A., Bronte, V., and Viola, A. (2011) Chemokine nitration prevents intratumoral infiltration of antigen-specific T cells. J. Exp. Med. 208, 1949-1962
42. Olivier, C., Oliver, L., Lalier, L., and Vallette, F. M. (2020) Drug Resistance in Glioblastoma: The Two Faces of Oxidative Stress. Front Mol Biosci 7, 620677
43. Cheng, G., Hardy, M., Topchyan, P., Zander, R., Volberding, P., Cui, W., and Kalyanaraman, B. (2020) Potent inhibition of tumour cell proliferation and immunoregulatory function by mitochondria-targeted atovaquone Sci. Rep. 10, 17872
44. Pan, J., Lee, Y., Cheng, G., Zielonka, J., Zhang, Q., Bajzikova, M., Xiong, D., Tsaih, S. W., Hardy, M., Flister, M., Olsen, C. M., Wang, Y., Vang, O., Neuzil, J., Myers, C. R., Kalyanaraman, B., and You, M. (2018) Mitochondria-Targeted Honokiol Confers a Striking Inhibitory Effect on Lung Cancer via Inhibiting Complex I Activity. iScience 3, 192-207
45. Cheng, G., Zielonka, J., Ouari, O., Lopez, M., McAllister, D., Boyle, K., Barrios, C. S., Weber, J. J., Johnson, B. D., Hardy, M., Dwinell, M. B., and Kalyanaraman, B. (2016) Mitochondria-Targeted Analogues of Metformin Exhibit Enhanced Antiproliferative and Radiosensitizing Effects in Pancreatic Cancer Cells. Cancer Res. 76, 3904-3915
46. Viswanadhan, V. N., Ghose, A. K., Revankar, G. R., and Robins, R. K. (1989) Atomic physicochemical parameters for three dimensional structure directed quantitative structure-activity relationships. 4. Additional parameters for hydrophobic and dispersive interactions and their application for an automated superposition of certain naturally occurring nucleoside antibiotics. Journal of Chemical Information and Computer Sciences 29, 163-172
47. Klopman, G., Li, J.-Y., Wang, S., and Dimayuga, M. (1994) Computer Automated log P Calculations Based on an Extended Group Contribution Approach. Journal of Chemical Information and Computer Sciences 34, 752-781
48. Bohle, D. S., Glassbrenner, P. A., and Hansert, B. (1996) Syntheses of pure tetramethylammonium peroxynitrite. Methods Enzymol. 269, 302-311
49. Szala, M., Grzelakowska, A., Modrzejewska, J., Siarkiewicz, P., Slowiński, D., Świerczyńska, M., Zielonka, J., and Podsiadly, R. (2020) Characterization of the reactivity of luciferin boronate-A probe for inflammatory oxidants with improved stability. Dyes and Pigments 183, 108693
50. Sikora, A., Zielonka, J., Lopez, M., Joseph, J., and Kalyanaraman, B. (2009) Direct oxidation of boronates by peroxynitrite: mechanism and implications in fluorescence imaging of peroxynitrite. Free Radical Biol. Med. 47, 1401-1407
51. Huang, M., Xiong, D., Pan, J., Zhang, Q., Wang, Y., Myers, C. R., Johnson, B. D., Hardy, M., Kalyanaraman, B., and You, M. (2022) Prevention of Tumor Growth and Dissemination by In Situ Vaccination with Mitochondria-Targeted Atovaquone. Advanced Science 9, 2101267
52. AbuEid, M., McAllister, D. M., McOlash, L., Harwig, M. C., Cheng, G., Drouillard, D., Boyle, K. A., Hardy, M., Zielonka, J., Johnson, B. D., Hill, R. B., Kalyanaraman, B., and Dwinell, M. B. (2021) Synchronous effects of targeted mitochondrial complex I inhibitors on tumor and immune cells abrogate melanoma progression. iScience 24, 102653
53. Zhang, Q., Xiong, D., Pan, J., Wang, Y., Hardy, M., Kalyanaraman, B., and You, M. (2022) Chemoprevention of Lung Cancer with a Combination of Mitochondria-Targeted Compounds. Cancers (Basel) 14, 2538
54. Corzo, C. A., Cotter, M. J., Cheng, P., Cheng, F., Kusmartsev, S., Sotomayor, E., Padhya, T., McCaffrey, T. V., McCaffrey, J. C., and Gabrilovich, D. I. (2009) Mechanism regulating reactive oxygen species in tumor-induced myeloid-derived suppressor cells. J. Immunol. 182, 5693-5701
55. Langley, M., Ghosh, A., Charli, A., Sarkar, S., Ay, M., Luo, J., Zielonka, J., Brenza, T., Bennett, B., Jin, H., Ghaisas, S., Schlichtmann, B., Kim, D., Anantharam, V., Kanthasamy, A., Narasimhan, B., Kalyanaraman, B., and Kanthasamy, A. G. (2017) Mito-Apocynin Prevents Mitochondrial Dysfunction, Microglial Activation, Oxidative Damage, and Progressive Neurodegeneration in MitoPark Transgenic Mice. Antioxid. Redox Signal. 27, 1048-1066
56. Ghosh, A., Langley, M. R., Harischandra, D. S., Neal, M. L., Jin, H., Anantharam, V., Joseph, J., Brenza, T., Narasimhan, B., Kanthasamy, A., Kalyanaraman, B., and Kanthasamy, A. G. (2016) Mitoapocynin Treatment Protects Against Neuroinflammation and Dopaminergic Neurodegeneration in a Preclinical Animal Model of Parkinson's Disease. J Neuroimmune Pharmacol 11, 259-278
57. Mukhopadhyay, P., Horvath, B., Zsengeller, Z., Zielonka, J., Tanchian, G., Holovac, E., Kechrid, M., Patel, V., Stillman, I. E., Parikh, S. M., Joseph, J., Kalyanaraman, B., and Pacher, P. (2012) Mitochondrial-targeted antioxidants represent a promising approach for prevention of cisplatin-induced nephropathy. Free Radic Biol Med 52, 497-506
58. Zielonka, J., Zielonka, M., VerPlank, L., Cheng, G., Hardy, M., Ouari, O., Ayhan, M. M., Podsiadly, R., Sikora, A., Lambeth, J. D., and Kalyanaraman, B. (2016) Mitigation of NADPH oxidase 2 activity as a strategy to inhibit peroxynitrite formation. J. Biol. Chem. 291, 7029-7044
59. Ford, K., Hanley, C. J., Mellone, M., Szyndralewiez, C., Heitz, F., Wiesel, P., Wood, O., Machado, M., Lopez, M. A., Ganesan, A. P., Wang, C., Chakravarthy, A., Fenton, T. R., King, E. V., Vijayanand, P., Ottensmeier, C. H., Al-Shamkhani, A., Savelyeva, N., and Thomas, G. J. (2020) NOX4 Inhibition Potentiates Immunotherapy by Overcoming Cancer-Associated Fibroblast-Mediated CD8 T-cell Exclusion from Tumors. Cancer Res. 80, 1846-1860
60. Konaté, M. M., Antony, S., and Doroshow, J. H. (2020) Inhibiting the Activity of NADPH Oxidase in Cancer. Antioxid. Redox Signal. 33, 435-454
61. Chung, A. W., Anand, K., Anselme, A. C., Chan, A. A., Gupta, N., Venta, L. A., Schwartz, M. R., Qian, W., Xu, Y., Zhang, L., Kuhn, J., Patel, T., Rodriguez, A. A., Belcheva, A., Darcourt, J., Ensor, J., Bernicker, E., Pan, P. Y., Chen, S. H., Lee, D. J., Niravath, P. A., and Chang, J. C. (2021) A phase 1/2 clinical trial of the nitric oxide synthase inhibitor L-NMMA and taxane for treating chemoresistant triple-negative breast cancer. Sci Transl Med 13, eabj5070
62. Adebayo, A. K., and Nakshatri, H. (2022) Modeling preclinical cancer studies under physioxia to enhance clinical translation. Cancer Res.
63. University of Oxford. (2019) Atovaquone as Tumour HypOxia Modifier (ATOM).
64. Skwarski, M., McGowan, D. R., Belcher, E., Di Chiara, F., Stavroulias, D., McCole, M., Derham, J. L., Chu, K. Y., Teoh, E., Chauhan, J., O'Reilly, D., Harris, B. H. L., Macklin, P. S., Bull, J. A., Green, M., Rodriguez-Berriguete, G., Prevo, R., Folkes, L. K., Campo, L., Ferencz, P., Croal, P. L., Flight, H., Qi, C., Holmes, J., O'Connor, J. P. B., Gleeson, F. V., McKenna, W. G., Harris, A. L., Bulte, D., Buffa, F. M., Macpherson, R. E., and Higgins, G. S. (2021) Mitochondrial Inhibitor Atovaquone Increases Tumor Oxygenation and Inhibits Hypoxic Gene Expression in Patients with Non-Small Cell Lung Cancer. Clin. Cancer. Res. 27, 2459-2469
65. Bourigault, P., Skwarski, M., Macpherson, R. E., Higgins, G. S., and McGowan, D. R. (2021) Investigation of atovaquone-induced spatial changes in tumour hypoxia assessed by hypoxia PET/CT in non-small cell lung cancer patients. EJNMMI Research 11, 130
66. Kalyanaraman, B., Cheng, G., and Hardy, M. (2022) Therapeutic Targeting of Tumor Cells and Tumor Immune Microenvironment Vulnerabilities. Front Oncol 12, 816504
67. Kalyanaraman, B. (2022) Exploiting the tumor immune microenvironment and immunometabolism using mitochondria-targeted drugs: Challenges and opportunities in racial disparity and cancer outcome research. FASEB J. 36, e22226
68. Piyarathna, D. W. B., Balasubramanian, A., Arnold, J. M., Lloyd, S. M., Karanam, B., Castro, P., Ittmann, M. M., Putluri, N., Navone, N., Jones, J. A., Yu, W., Sandulache, V. C., Sikora, A. G., Michailidis, G., and Sreekumar, A. (2019) ERRI and PGC1α associated mitochondrial alterations correlate with pan-cancer disparity in African Americans. J. Clin. Invest. 129, 2351-2356
69. Choudhury, A. R., and Singh, K. K. (2017) Mitochondrial determinants of cancer health disparities. Semin. Cancer Biol. 47, 125-146
70. Reddy, K. R. K., Park, J. H., Bollag, R. J., Bellman, A., Terris, M., Lerner, S. P., Ballester, L. Y., Lotan, Y., Kaipparettu, B. A., and Putluri, N. (2022) Abstract 3771: Mitochondrial metabolism and racial disparity of bladder cancer. Cancer Res. 82, 3771-3771
71. Kim, G., Pastoriza, J. M., Condeelis, J. S., Sparano, J. A., Filippou, P. S., Karagiannis, G. S., and Oktay, M. H. (2020) The Contribution of Race to Breast Tumor Microenvironment Composition and Disease Progression. Front Oncol 10, 1022
72. Yaghoobi, V., Moutafi, M., Aung, T. N., Pelekanou, V., Yaghoubi, S., Blenman, K., Ibrahim, E., Vathiotis, I. A., Shafi, S., Sharma, A., O'Meara, T., Fernandez, A. I., Pusztai, L., and Rimm, D. L. (2021) Quantitative assessment of the immune microenvironment in African American Triple Negative Breast Cancer: a case-control study. Breast Cancer Res 23, 113
73. Abdou, Y., Attwood, K., Cheng, T. D., Yao, S., Bandera, E. V., Zirpoli, G. R., Ondracek, R. P., Stein, L., Bshara, W., Khoury, T., Ambrosone, C. B., and Omilian, A. R. (2020) Racial differences in CD8 (+) T cell infiltration in breast tumors from Black and White women. Breast Cancer Res 22, 62
74. Yao, S., Cheng, T. D., Elkhanany, A., Yan, L., Omilian, A., Abrams, S.

I., Evans, S., Hong, C. C., Qi, Q., Davis, W., Liu, S., Bandera, E. V., Odunsi, K., Takabe, K., Khoury, T., and Ambrosone, C. B. (2021) Breast Tumor Microenvironment in Black Women: A Distinct Signature of CD8+ T-Cell Exhaustion. J. Natl. Cancer Inst. 113, 1036-1043
75. Beebe-Dimmer, J. L., and Cooney, K. A. (2019) Mitochondrial alterations may underlie race-specific differences in cancer risk and outcome. J. Clin. Invest. 129, 2187-2188
76. Williams, L. K., Padhukasahasram, B., Ahmedani, B. K., Peterson, E. L., Wells, K. E., González Burchard, E., and Lanfear, D. E. (2014) Differing effects of metformin on glycemic control by race-ethnicity. J. Clin. Endocrinol. Metab. 99, 3160-3168
77. Weinberg, S. E., and Chandel, N. S. (2015) Targeting mitochondria metabolism for cancer therapy. Nat. Chem. Biol. 11, 9-15
78. Vyas, S., Zaganjor, E., and Haigis, M. C. (2016) Mitochondria and cancer. Cell 166, 555-566
79. Xu, Y., Xue, D., Bankhead, A., 3rd, and Neamati, N. (2020) Why All the Fuss about Oxidative Phosphorylation (OXPHOS)? J. Med. Chem. 63, 14276-14307
80. Chandel, N. S. (2018) Mitochondria: back to the future. Nat. Rev. Mol. Cell Biol. 19, 76
81. Cheng, G., Hardy, M., You, M., and Kalyanaraman, B. (2022) Combining PEGylated mito-atovaquone with MCT and Krebs cycle redox inhibitors as a potential strategy to abrogate tumor cell proliferation. Sci Rep 12, 5143
82. Evans, K. W., Yuca, E., Scott, S. S., Zhao, M., Paez Arango, N., Cruz Pico, C. X., Saridogan, T., Shariati, M., Class, C. A., Bristow, C. A., Vellano, C. P., Zheng, X., Gonzalez-Angulo, A. M., Su, X., Tapia, C., Chen, K., Akcakanat, A., Lim, B., Tripathy, D., Yap, T. A., Francesco, M. E. D., Draetta, G. F., Jones, P., Heffernan, T. P., Marszalek, J. R., and Meric-Bernstam, F. (2021) Oxidative Phosphorylation Is a Metabolic Vulnerability in Chemotherapy-Resistant Triple-Negative Breast Cancer. Cancer Res. 81, 5572-5581
83. Cheng, G., Hardy, M., Zielonka, J., Weh, K. M., Zielonka, M., Boyle, K., Abu Eid, M., McAllister, D., Bennett, B., Kresty, L. A., Dwinell, M., and Kalyanaraman, B. (2020) Mitochondria-Targeted Magnolol Inhibits OXPHOS, Proliferation, and Tumor Growth via Modulation of Energetics and Autophagy in Melanoma Cells. Cancer Research and Treatment Communications 25, 100210

For reasons of completeness, various aspects of the invention are set out in the following numbered clauses:
Clause 1. A compound of formula (I) or (II),

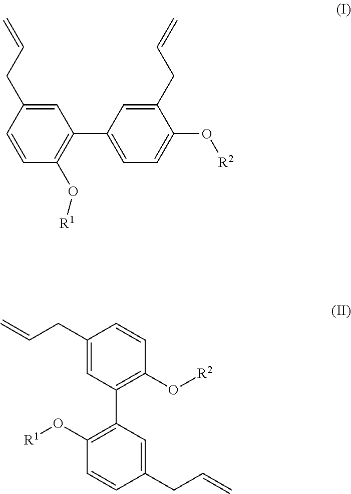

wherein
one of R¹ and R² is H,

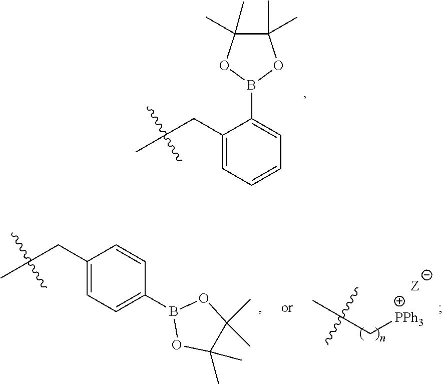

the other of R¹ and R² is

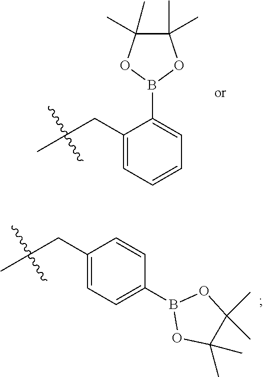 [SMILES: *Cc1ccccc1B1OC(C)(C)C(C)(C)O1 ; *Cc1ccc(B2OC(C)(C)C(C)(C)O2)cc1]

n is an integer from 1-20; and
Z^{⊖} is a counterion.
Clause 2. The compound of clause 1, which is a compound of formula (I).
Clause 3. The compound of clause 1, which is a compound of formula (II).
Clause 4. The compound of any one of clauses 1-3, wherein
one of R¹ and R² is

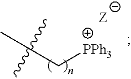

and
the other of R¹ and R² is

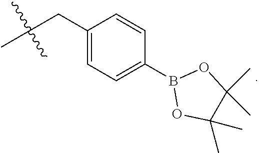 [SMILES: *Cc1ccc(B2OC(C)(C)C(C)(C)O2)cc1]

Clause 5. The compound of any one of clauses 1-4, wherein n is 10.
Clause 6. The compound of any one of clauses 1-5, wherein Z is Br.
Clause 7. The compound of clause 1, wherein the compound is

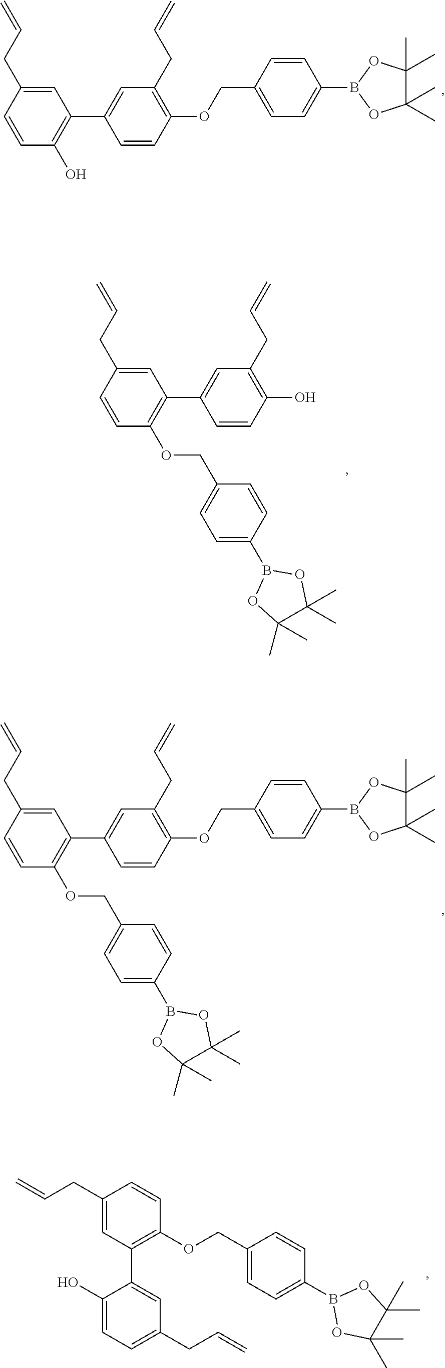

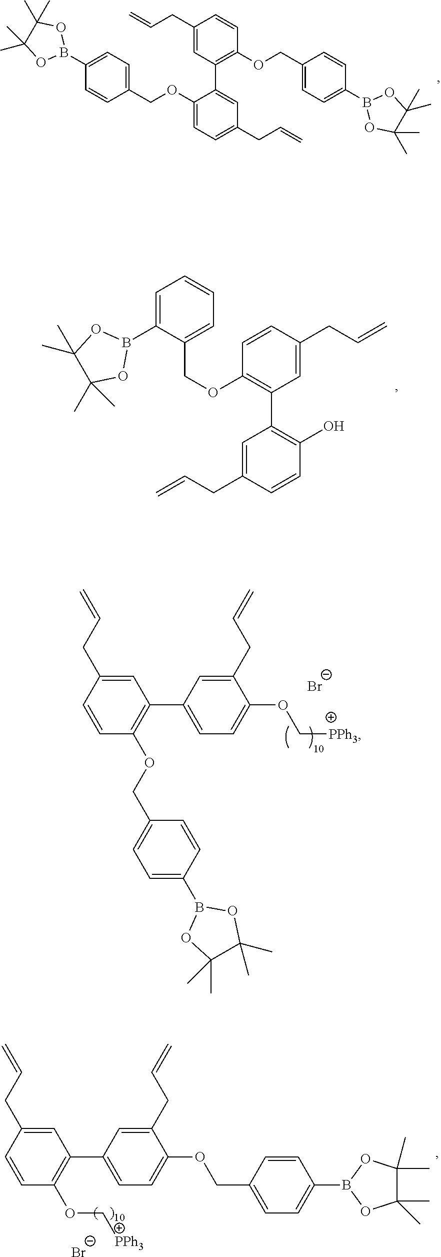

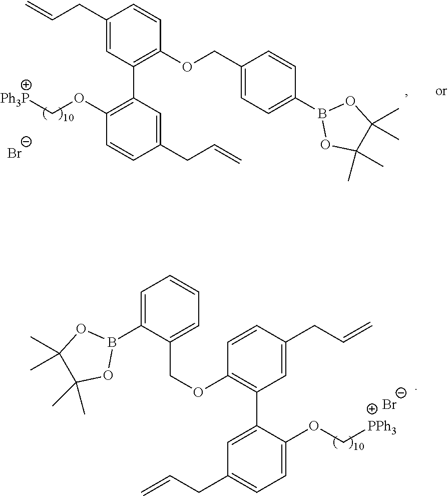

Clause 8. The compound of clause 1, wherein the compound is

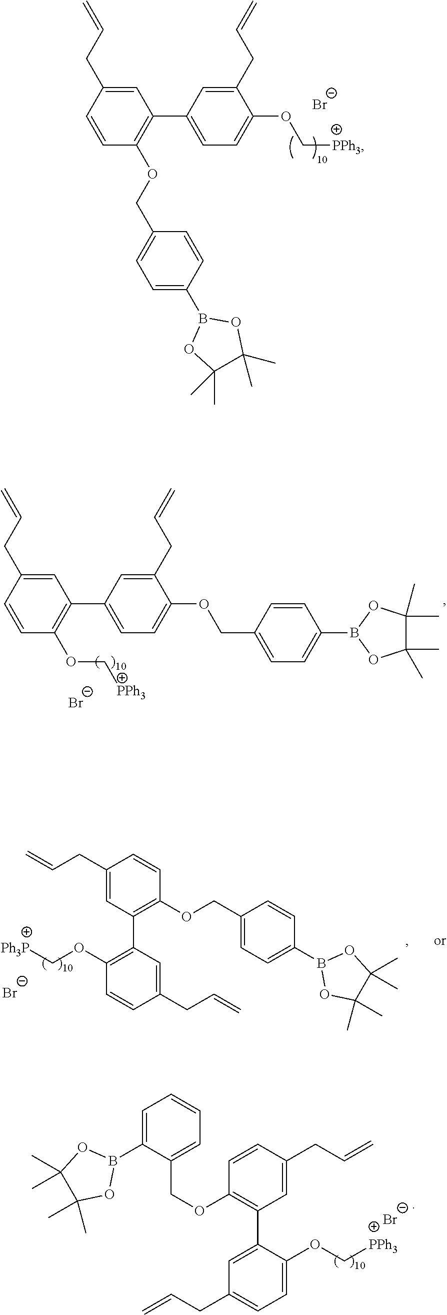

Clause 9. A pharmaceutical composition comprising the compound of any one of clauses 1-8 and a pharmaceutically acceptable carrier, diluent, or excipient.
Clause 10. A method of treating cancer in a subject in need thereof, the method comprising administering a therapeutically effective amount of the compound of any one of clauses 1-8 or the pharmaceutical composition of clause 9 to the subject.
Clause 11. The method of clause 10, further comprising treating the subject with boron neutron capture therapy (BNCT) using the administered compound or composition, thereby the cancer is treated.
Clause 12. The method of clause 11, wherein the compound or composition is targeted to cancer cell mitochondria.
Clause 13. A method of reducing reactive oxygen and nitrogen species in tumor microenvironment of a cancer in a subject in need thereof, the method comprising administering an effective amount of the compound of any one of clauses 1-8 or the pharmaceutical composition of clause 9 to the subject.
Clause 14. The method of clause 13, wherein a NADPH oxidase (NOX) or an inducible nitric oxide synthase (iNOS) in the tumor microenvironment is inhibited or nitric oxide level in the tumor microenvironment is reduced, thereby reducing inflammation in the subject.
Clause 15. The method of clause 14, wherein nitric oxide level in the tumor microenvironment is reduced, and wherein the nitric oxide comprises ONOO.
Clause 16. A method of analyzing reactive oxygen or nitrogen species in tumor microenvironment of a cancer, the method comprising:
introducing the compound of any one of clauses 1-8 or the pharmaceutical composition of clause 9 to the tumor microenvironment, whereby the reactive oxygen or nitrogen species reacts with the compound or composition to produce a product; and
detecting the product thereby analyzing the reactive oxygen or nitrogen species in the tumor microenvironment.
Clause 17. A method of reducing or inhibiting cancer cell growth in a subject in need thereof, the method comprising administering a therapeutically effective amount of the compound of any one of clauses 1-8 or the pharmaceutical composition of clause 9 to the subject.
Clause 18. The method of clause 17, wherein T cell killing of cancer cells is enhanced.
Clause 19. The method of clause 17, further comprising treating the subject with boron neutron capture therapy (BNCT) using the administered compound or composition, thereby cancer cell growth is reduced or inhibited.
Clause 20. The method of clause 17, wherein the compound or composition is targeted to cancer cell mitochondria.
Clause 21. The compound of any one of clauses 1-8 or the pharmaceutical composition of clause 9 for use in therapy.
Clause 22. The compound of any one of clauses 1-8 or the pharmaceutical composition of clause 9 for use in treatment of cancer.
Clause 23. The compound of any one of clauses 1-8 or the pharmaceutical composition of clause 9 for use in boron neutron capture therapy (BNCT) for cancer.
Clause 24. The compound or composition of clause 23, wherein the compound or composition is targeted to cancer cell mitochondria.
Clause 25. Use of a compound according to any one of clauses 1-8 for the manufacture of a medicament for the treatment of cancer.
Clause 26. The method of any one of clauses 10-20, the compound or composition of any one of clauses 22-24, or the use of clause 25, wherein the cancer is breast cancer, colon cancer, gastric cancer, glioblastoma, head and neck cancer, kidney cancer, liver cancer, lung cancer, melanoma, ovarian cancer, pancreatic cancer, prostate cancer, or a combination thereof.
Clause 27. A kit comprising the pharmaceutical composition of clause 9 and instructional material.

Although the invention has been described in considerable detail with reference to certain embodiments, one skilled in the art will appreciate that the present invention can be used in alternative embodiments to those described, which have been presented for purposes of illustration and not of limitation. Therefore, the scope of the appended claims should not be limited to the description of the embodiments contained herein.

1. A compound of formula (I) or (II), 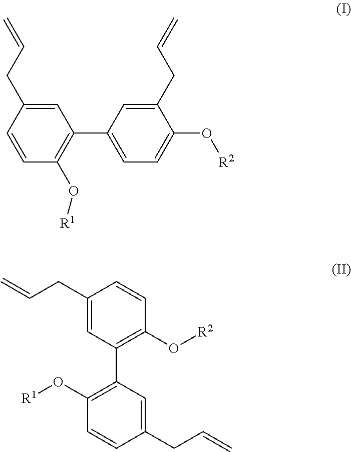
wherein
one of R¹ and R² is H, 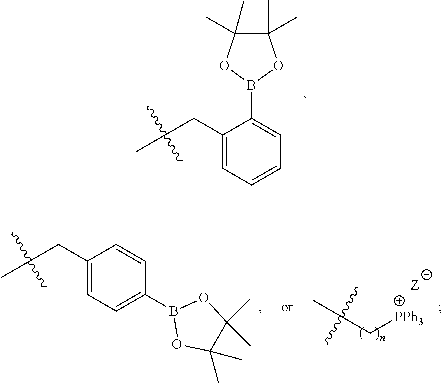
the other of R¹ and R² is 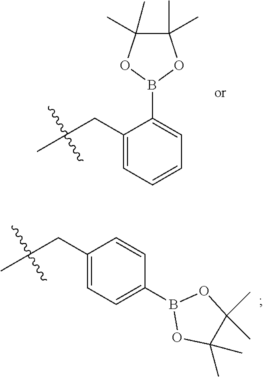 [SMILES: *Cc1ccccc1B1OC(C)(C)C(C)(C)O1 ; *Cc1ccc(B2OC(C)(C)C(C)(C)O2)cc1]
n is an integer from 1-20; and
Z^{⊖} is a counterion.

2. The compound of claim 1, which is a compound of formula (I).

3. The compound of claim 1, which is a compound of formula (II).

4. The compound of claim 1, wherein
one of R¹ and R² is 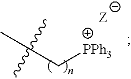
and
the other of R¹ and R² is

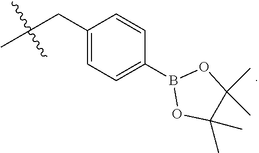 [SMILES: *Cc1ccc(B2OC(C)(C)C(C)(C)O2)cc1]

5. The compound of claim 1, wherein n is 10.

6. The compound of claim 1, wherein Z is Br.

7. The compound of claim 1, wherein the compound is

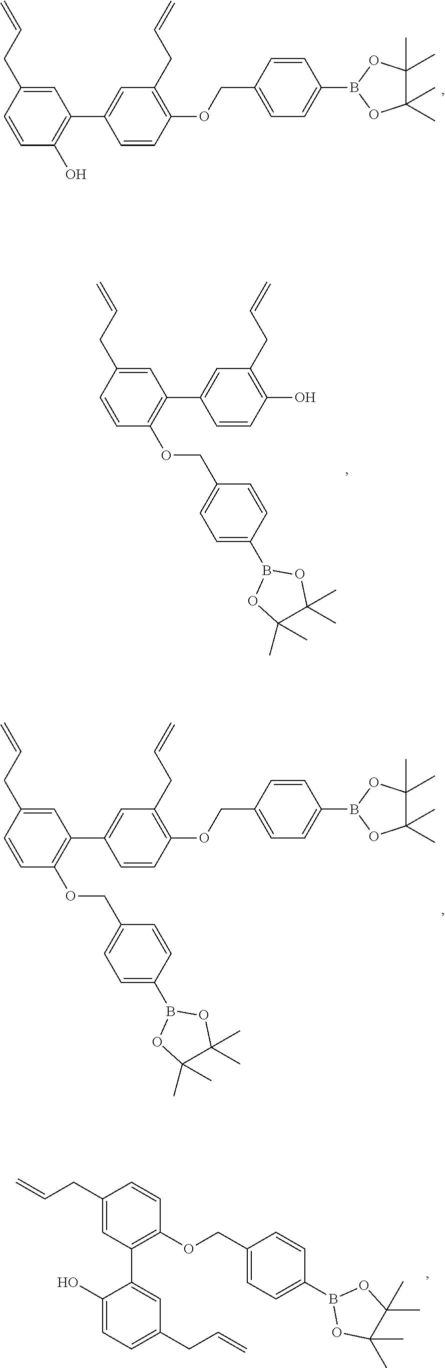

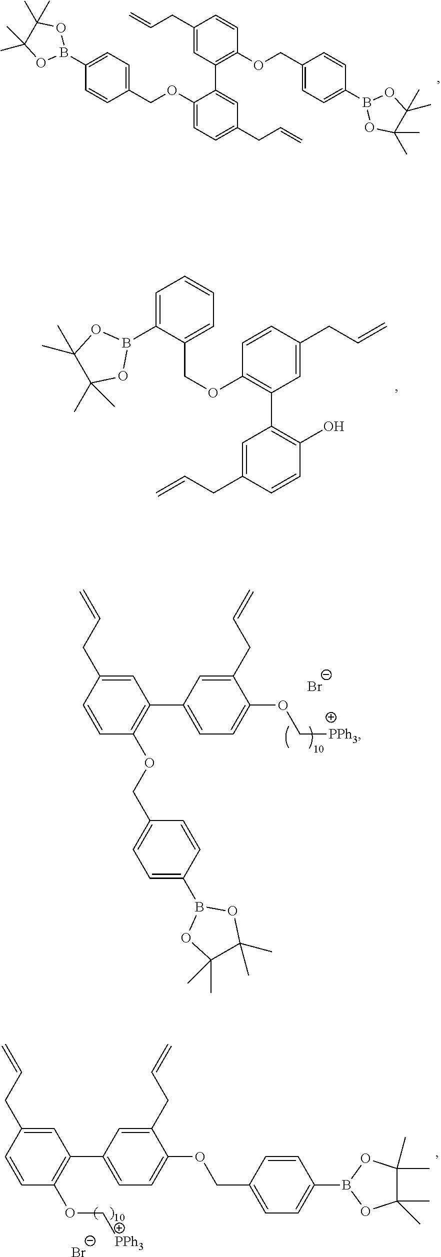

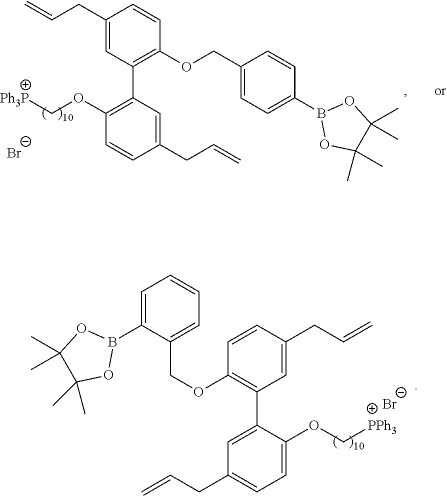

8. The compound of claim 1, wherein the compound is

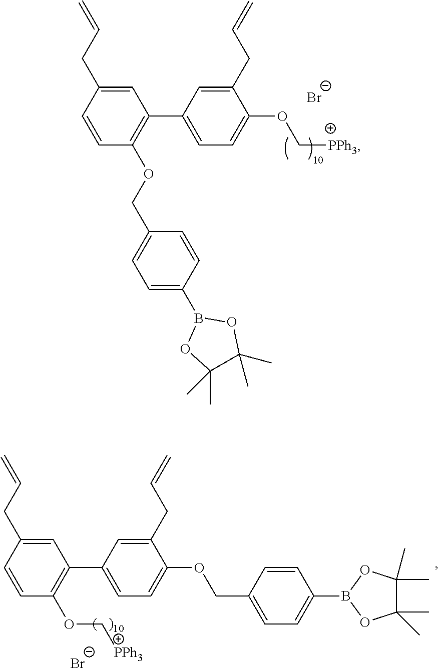

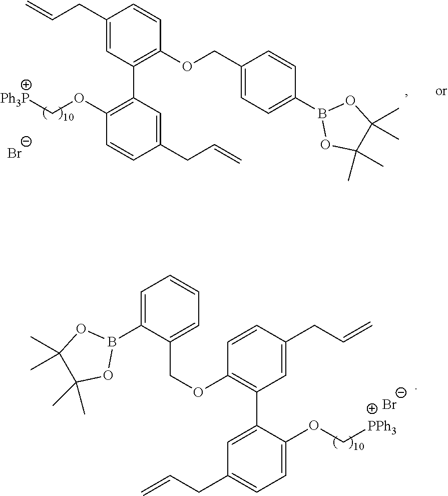

9. A pharmaceutical composition comprising the compound of claim 1 and a pharmaceutically acceptable carrier, diluent, or excipient.

10. A method of treating cancer in a subject in need thereof, the method comprising administering a therapeutically effective amount of the compound of claim 1 to the subject.

11. The method of claim 10, further comprising treating the subject with boron neutron capture therapy (BNCT) using the administered compound or composition, thereby the cancer is treated.

12. (canceled)

13. A method of reducing reactive oxygen and nitrogen species in tumor microenvironment of a cancer in a subject in need thereof, the method comprising administering an effective amount of the compound of claim 1 to the subject.

14. The method of claim 13, wherein a NADPH oxidase (NOX) or an inducible nitric oxide synthase (iNOS) in the tumor microenvironment is inhibited or nitric oxide level in the tumor microenvironment is reduced, thereby reducing inflammation in the subject.

15. The method of claim 14, wherein nitric oxide level in the tumor microenvironment is reduced, and wherein the nitric oxide comprises ONOO⁻.

16. A method of analyzing reactive oxygen or nitrogen species in tumor microenvironment of a cancer, the method comprising:
introducing the compound of claim 1 to the tumor microenvironment, whereby the reactive oxygen or nitrogen species reacts with the compound or composition to produce a product; and
detecting the product thereby analyzing the reactive oxygen or nitrogen species in the tumor microenvironment.

17. A method of reducing or inhibiting cancer cell growth in a subject in need thereof, the method comprising administering a therapeutically effective amount of the compound of claim 1 to the subject.

18. The method of claim 17, wherein T cell killing of cancer cells is enhanced.

19. The method of claim 17, further comprising treating the subject with boron neutron capture therapy (BNCT) using the administered compound or composition, thereby cancer cell growth is reduced or inhibited.

20-25. (canceled)

26. The method of claim 10, wherein the cancer is breast cancer, colon cancer, gastric cancer, glioblastoma, head and neck cancer, kidney cancer, liver cancer, lung cancer, melanoma, ovarian cancer, pancreatic cancer, prostate cancer, or a combination thereof.

27. A kit comprising the pharmaceutical composition of claim 9 and instructional material.
